(19) United States
(12) Patent Application Publication
(10) Pub. No.: US 2006/0247305 A1
(43) Pub. Date: Nov. 2, 2006

(54) Chromen-4-one inhibitors of anti-apoptotic Bcl-2 family members and the uses thereof

(75) Inventors: Shaomeng Wang, Saline, MI (US); Ke Ding, Guangzhou (CN); Guozhi Tang, Ann Arbor, MI (US); Zaneta Nikolovska-Coleska, Ann Arbor, MI (US); Renxio Wang, Ann Arbor, MI (US); Chao-Yie Yang, Ann Arbor, MI (US)

Correspondence Address: Medlen & Carroll, LLP, Suite 350, 101 Howard Street, San Francisco, CA, 94105, US

(73) Assignee: Regents of the University of Michigan, Ann Arbor, MI (US)

(21) Appl. No.: 11/373,898

(22) Filed: Mar. 13, 2006

Related U.S. Application Data
(60) Provisional application No. 60/661,265, filed on Mar. 11, 2005.

(51) Int. Cl. A61K 31/353; C07D 405/14

(52) U.S. Cl. 514/456; 549/403

(57) ABSTRACT

The invention relates to small molecules which function as inhibitors of anti-apoptotic Bcl-2 family member proteins (e.g., Bcl-2 and Bcl-xL). The invention also relates to the use of these compounds for inducing apoptotic cell death and sensitizing cells to the induction of apoptotic cell death.

The present application claims priority to U.S. Provisional Application Ser. No. 60/661,265, filed Mar. 11, 2005, herein incorporated by reference.

### BACKGROUND OF THE INVENTION

1. Field of the Invention

This invention is in the field of medicinal chemistry. In particular, the invention relates to small molecules which function as inhibitors of anti-apoptotic Bcl-2 family member proteins (e.g., Bcl-2 and Bcl-xL). The invention also relates to the use of these compounds for inducing apoptotic cell death and sensitizing cells to the induction of apoptotic cell death.

2. Related Art

The aggressive cancer cell phenotype is the result of a variety of genetic and epigenetic alterations leading to deregulation of intracellular signaling pathways (Ponder, Nature 411:336 (2001)). The commonality for all cancer cells, however, is their failure to execute an apoptotic program, and lack of appropriate apoptosis due to defects in the normal apoptosis machinery is a hallmark of cancer (Lowe et al., Carcinogenesis 21:485 (2000)). Most of the current cancer therapies, including chemotherapeutic agents, radiation, and immunotherapy, work by indirectly inducing apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Primary or acquired resistance of human cancer of different origins to current treatment protocols due to apoptosis defects is a major problem in current cancer therapy (Lowe et al., Carcinogenesis 21:485 (2000); Nicholson, Nature 407:810 (2000)). Accordingly, current and future efforts towards designing and developing new molecular target-specific anticancer therapies to improve survival and quality of life of cancer patients must include strategies that specifically target cancer cell resistance to apoptosis. In this regard, targeting crucial negative regulators that play a central role in directly inhibiting apoptosis in cancer cells represents a highly promising therapeutic strategy for new anticancer drug design.

Two classes of central negative regulators of apoptosis have been identified. The first class of negative regulators of apoptosis is the inhibitor of apoptosis proteins (IAPs) (Deveraux et al., Genes Dev. 13:239 (1999); Salvesen et al., Nat. Rev. Mol. Cell. Biol. 3:401 (2002)). IAP proteins potently suppress apoptosis induced by a large variety of apoptotic stimuli, including chemotherapeutic agents, radiation, and immunotherapy in cancer cells.

The second class of central negative regulators of apoptosis is the Bcl-2 family of proteins (Adams et al., Science 281:1322 (1998); Reed, Adv. Pharmacol. 41:501 (1997); Reed et al., J. Cell. Biochem. 60:23 (1996)). Bcl-2 is the founding member of the family and was first isolated as the product of an oncogene. The Bcl-2 family now includes both anti-apoptotic molecules such as Bcl-2, Bcl-xL, and Mcl-1 and pro-apoptotic molecules such as Bax, Bak, Bid, and Bad. Bcl-2, Bcl-xL, and Mcl-1 are overexpressed in many types of human cancer (e.g., breast, prostate, colorectal, lung), including Non-Hodgkin's lymphoma, which is caused by a chromosomal translocation (t14, 18) that leads to overexpression of Bcl-2. This suggests that many cancer cell types depend on the elevated levels of Bcl-2 family proteins to survive the other cellular derangements that simultaneously both define them as cancerous or pre-cancerous cells and cause them to attempt to execute the apoptosis pathway. Also, increased expression of Bcl-2 family proteins has been recognized as a basis for the development of resistance to cancer therapeutic drugs and radiation that act in various ways to induce cell death in tumor cells.

Bcl-2 and Bcl-xL are thought to play a role in tumor cell migration and invasion, and therefore, metastasis. Amberger et al., Cancer Res. 58:149 (1998); Wick et al., FEBS Lett, 440:419 (1998); Mohanam et al., Cancer Res. 53:4143 (1993); Pedersen et al., Cancer Res., 53:5158 (1993). Bcl-2 family proteins appear to provide tumor cells with a mechanism for surviving in new and non-permissive environments (e.g., metastatic sites), and contribute to the organospecific pattern of clinical metastatic cancer spread. Rubio, Lab Invest. 81:725 (2001); Fernández et al., Cell Death Differ. 7:350 (2000)). Anti-apoptotic proteins such as Bcl-2 and/or Bcl-xL are also thought to regulate cell-cell interactions, for example through regulation of cell surface integrins. Reed, Nature 387:773 (1997); Frisch et al., Curr. Opin. Cell Biol. 9:701 (1997); Del Bufalo et al., FASEB J. 11:947 (1997).

Therapeutic strategies for targeting Bcl-2, Bcl-xL, and Mcl-1 in cancer to restore cancer cell sensitivity and overcome resistance of cancer cells to apoptosis have been extensively reviewed (Adams et al., Science 281:1322 (1998); Reed, Adv. Pharmacol. 41:501 (1997); Reed et al., J. Cell. Biochem. 60:23 (1996)). Currently, Bcl-2 antisense therapy is in several Phase III clinical trials for the treatment of solid and non-solid tumors.

Gossypol is a naturally occurring double biphenolic compound derived from crude cotton seed oil (Gossypium sp.). Human trials of gossypol as a male contraceptive have demonstrated the safety of long term administration of these compounds (Wu, Drugs 38:333 (1989)). Gossypol has more recently been shown to have some anti-proliferative effects (Flack et al., J. Clin. Endocrinol. Metab. 76:1019 (1993); Bushunow et al., J. Neuro-Oncol. 43:79, (1999); Van Poznak et al., Breast Cancer Res. Treat. 66:239 (2001)). (−)-Gossypol and its derivatives recently have been shown to be potent inhibitors of Bcl-2, Bcl-xL, and Mcl-1 and to have strong anti-cancer activity (U.S. Patent Application Nos. 2003/0008924; 2004/0214902).

### SUMMARY OF THE INVENTION

It is generally accepted that the inability of cancer cells or their supporting cells to undergo apoptosis in response to genetic lesions or exposure to inducers of apoptosis (such as anticancer agents and radiation) is a major factor in the onset and progression of cancer. The induction of apoptosis in cancer cells or their supporting cells (e.g., neovascular cells in the tumor vasculature) is thought to be a universal mechanism of action for virtually all of the effective cancer therapeutic drugs or radiation therapies on the market or in practice today. One reason for the inability of a cell to undergo apoptosis is increased expression and accumulation of anti-apoptotic. Bcl-2 family members.

The present invention contemplates that exposure of animals suffering from cancer to therapeutically effective amounts of drug(s) (e.g., small molecules) that inhibit the function(s) of anti-apoptotic Bcl-2 family members will kill cancer cells or supporting cells outright (those cells whose continued survival is dependent on the overactivity of anti-apoptotic Bcl-2 family members) and/or render such cells as a population more susceptible to the cell death-inducing activity of cancer therapeutic drugs or radiation therapies. The present invention contemplates that inhibitors of anti-apoptotic Bcl-2 family members satisfy an unmet need for the treatment of multiple cancer types, either when administered as monotherapy to induce apoptosis in cancer cells dependent on anti-apoptotic Bcl-2 family member function, or when administered in a temporal relationship with other cell death-inducing cancer therapeutic drugs or radiation therapies so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of a compound of the present invention and a course of an anticancer agent or radiation produces a greater tumor response and clinical benefit in such animals compared to those treated with the compound or anticancer drugs/radiation alone. Put another way, because the compounds lower the apoptotic threshold of all cells that express anti-apoptotic Bcl-2 family members, the proportion of cells that successfully execute the apoptosis program in response to the apoptosis inducing activity of anticancer drugs/radiation is increased. Alternatively, the compounds of the present invention can be used to allow administration of a lower, and therefore less toxic and more tolerable, dose of an anticancer agent and/or radiation to produce the same tumor response/clinical benefit as the conventional dose of the anticancer agent/radiation alone. Since the doses for all approved anticancer drugs and radiation treatments are known, the present invention contemplates the various combinations of them with the present compounds. Also, since the compounds of the present invention may act at least in part by inhibiting anti-apoptotic Bcl-2 family members, the exposure of cancer cells and supporting cells to therapeutically effective amounts of the compounds should be temporally linked to coincide with the attempts of cells to execute the apoptosis program in response to the anticancer agent or radiation therapy. Thus, in some embodiments, administering the compositions of the present invention in connection with certain temporal relationships, provides especially efficacious therapeutic practices.

The present invention relates to compounds that are useful for inhibiting the activity of anti-apoptotic Bcl-2 family members and increasing the sensitivity of cells to inducers of apoptosis. In one particular embodiment, the compounds have formula I: 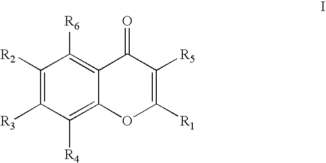
or a pharmaceutically acceptable salt or prodrug thereof, wherein:
R₁ is H, OH, F, Cl, Br, I, or optionally substituted alkyl, cycloalkyl, alkenyl, cycloalkenyl, alkynyl, aryl, heteroaryl, or heterocyclic;
R₂, R₃, R₄, R₅, and R₆ are independently H, F, Cl, Br, I, OH, or optionally substituted alkyl, cycloalkyl, alkenyl, cycloalkenyl, alkynyl, aryl, heteroaryl, heterocyclic, CO₂R′, C(O)NR′R″, SO₂NR′R″, SR′, OR′, NR″C(O)R′, NR′SO₂R″, or NR′R″;
R′ and R″ are independently H or optionally substituted alkyl, cycloalkyl, alkenyl, cycloalkenyl, alkynyl, aryl, heteroaryl, or heterocyclic, or R′ and R″ together with the N to which they are attached form a heterocyclic or heteroaryl ring.

In one embodiment, the compounds of Formula I have Formula II: 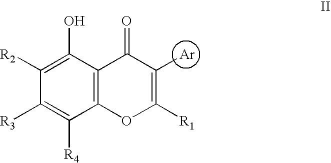
or a pharmaceutically acceptable salt or prodrug thereof, wherein:
Ar is optionally substituted aryl or heteroaryl.

In another embodiment, the compounds of Formula I have Formula III: 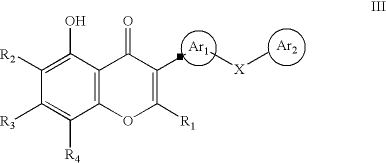
or a pharmaceutically acceptable salt or prodrug thereof, wherein:
Ar₁ and Ar₂ are independently optionally substituted aryl or heteroaryl;
X is O, NR′, SO₂, S, C(O)N(R′), SO₂NR′, R′NCO, R′NSO₂, N(R′)R″, N(R′)—R″—N(R′″), R′, OR′, OR′O, or C(O)N(R′)R″; and
R′, R″, and R′″ are independently H or optionally substituted alkyl, cycloalkyl, alkenyl, cycloalkenyl, alkynyl, aryl, heteroaryl, or heterocyclic,
or two of R′, R″, and R′″ form a heterocyclic or heteroaryl ring.

In another embodiment, the compounds of Formula I have Formula IV: 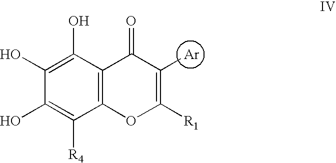
or a pharmaceutically acceptable salt or prodrug thereof.

In another embodiment, the compounds of Formula I have Formula V: 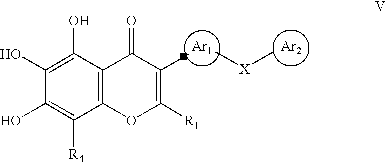
or a pharmaceutically acceptable salt or prodrug thereof.

In another embodiment, the compounds of Formula I have Formula VI: 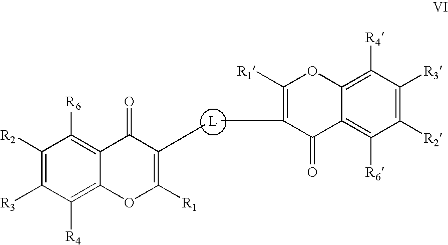
or a pharmaceutically acceptable salt or prodrug thereof; wherein
L is optionally substituted aryl, bi-aryl, heteroaryl, heterocyclic, alkyl, cycloalkyl, alkenyl, cycloalkenyl, alkynyl, ether, ester, amine, amide, sulfonyl, sulfonamide, or thioether;
R₁ and R₁′ are independently H, OH, F, Cl, Br, I, or optionally substituted alkyl, cycloalkyl, alkenyl, cycloalkenyl, alkynyl, aryl, heteroaryl, or heterocyclic; and R₂, R₂′, R₃, R₃′, R₄, R₄′, R₆, and R₆′ are independently H, F, Cl, Br, I, OH, or optionally substituted alkyl, cycloalkyl, alkenyl, cycloalkenyl, alkynyl, aryl, heteroaryl, heterocyclic, CO₂R′, C(O)NR′R″, SO₂NR′R″, SR′, OR′, NR″C(O)R′, NR′SO₂R″, or NR′R″.

The invention relates to compounds represented by Formula I, which are inhibitors of anti-apoptotic Bcl-2 family members. The invention relates to the use of the compounds of the invention to induce apoptosis in cells. The invention also relates to the use of the compounds of the invention for sensitizing cells to inducers of apoptosis. The compounds are useful for the treatment, amelioration, or prevention of disorders responsive to induction of apoptotic cell death, e.g., disorders characterized by dysregulation of apoptosis, including hyperproliferative diseases such as cancer. In certain embodiments, the compounds can be used to treat, ameliorate, or prevent cancer that is characterized by resistance to cancer therapies (e.g., those which are chemoresistant, radiation resistant, hormone resistant, and the like). In other embodiments, the compounds can be used to treat hyperproliferative diseases characterized by overexpression of anti-apoptotic Bcl-2 family members.

The present invention provides pharmaceutical compositions comprising a compound of Formula I in a therapeutically effective amount to induce apoptosis in cells or to sensitize cells to inducers of apoptosis.

The invention further provides kits comprising a compound of Formula I and instructions for administering the compound to an animal. The kits may optionally contain other therapeutic agents, e.g., anticancer agents, apoptosis modulating agents.

The invention also provides methods of making compounds of Formula I.

### BRIEF DESCRIPTION OF THE DRAWINGS/FIGURES

FIG. 1 shows the interactions between gossypol and Bcl-xL.

### DETAILED DESCRIPTION OF THE INVENTION

The present invention relates to compounds represented by Formula I, which function as inhibitors of anti-apoptotic Bcl-2 family members. By inhibiting anti-apoptotic Bcl-2 family members, these compounds sensitize cells to inducers of apoptosis and, in some instances, themselves induce apoptosis. Therefore, the invention relates to methods of sensitizing cells to inducers of apoptosis and to methods of inducing apoptosis in cells, comprising contacting the cells with a compound of Formula I alone or in combination with an inducer of apoptosis. The invention further relates to methods of treating, ameliorating, or preventing disorders in an animal that are responsive to induction of apoptosis comprising administering to the animal a compound of Formula I and an inducer of apoptosis. Such disorders include those characterized by a dysregulation of apoptosis and those characterized by overexpression of anti-apoptotic Bcl-2 family members.

The term “anti-apoptotic Bcl-2 family members,” as used herein, refers to any known member of the Bcl-2 family of proteins which has anti-apoptotic activity, including, but not limited to, Bcl-2, Bcl-xL, Mcl-1, A1/BFL-1, BOO-DIVA, Bcl-w, Bcl-6, Bcl-8 and Bcl-y.

The term “overexpression of anti-apoptotic Bcl-2 family members,” as used herein, refers to an elevated level (e.g., aberrant level) of mRNAs encoding for an anti-apoptotic Bcl-2 family member protein(s), and/or to elevated levels of anti-apoptotic Bcl-2 family member protein(s) in cells as compared to similar corresponding non-pathological cells expressing basal levels of mRNAs encoding anti-apoptotic Bcl-2 family member proteins or having basal levels of anti-apoptotic Bcl-2 family member proteins. Methods for detecting the levels of mRNAs encoding anti-apoptotic Bcl-2 family member proteins or levels of anti-apoptotic Bcl-2 family member proteins in a cell include, but are not limited to, Western blotting using anti-apoptotic Bcl-2 family member protein antibodies, immunohistochemical methods, and methods of nucleic acid amplification or direct RNA detection. As important as the absolute level of anti-apoptotic Bcl-2 family member proteins in cells is to determining that they overexpress anti-apoptotic Bcl-2 family member proteins, so also is the relative level of anti-apoptotic Bcl-2 family member proteins to other pro-apoptotic signaling molecules (e.g., pro-apoptotic Bcl-2 family proteins) within such cells. When the balance of these two are such that, were it not for the levels of the anti-apoptotic Bcl-2 family member proteins, the pro-apoptotic signaling molecules would be sufficient to cause the cells to execute the apoptosis program and die, said cells would be dependent on the anti-apoptotic Bcl-2 family member proteins for their survival. In such cells, exposure to an inhibiting effective amount of an anti-apoptotic Bcl-2 family member protein inhibitor will be sufficient to cause the cells to execute the apoptosis program and die. Thus, the term “overexpression of an anti-apoptotic Bcl-2 family member protein” also refers to cells that, due to the relative levels of pro-apoptotic signals and anti-apoptotic signals, undergo apoptosis in response to inhibiting effective amounts of compounds that inhibit the function of anti-apoptotic Bcl-2 family member proteins.

The terms “anticancer agent” and “anticancer drug,” as used herein, refer to any therapeutic agents (e.g., chemotherapeutic compounds and/or molecular therapeutic compounds), radiation therapies, or surgical interventions, used in the treatment of hyperproliferative diseases such as cancer (e.g., in mammals).

The term “prodrug,” as used herein, refers to a pharmacologically inactive derivative of a parent “drug” molecule that requires biotransformation (e.g., either spontaneous or enzymatic) within the target physiological system to release, or to convert (e.g., enzymatically, mechanically, electromagnetically) the prodrug into the active drug. Prodrugs are designed to overcome problems associated with stability, toxicity, lack of specificity, or limited bioavailability. Exemplary prodrugs comprise an active drug molecule itself and a chemical masking group (e.g., a group that reversibly suppresses the activity of the drug). Some preferred prodrugs are variations or derivatives of compounds that have groups cleavable under metabolic conditions. Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of parent acids with a suitable alcohol (e.g., a lower alkanol), amides prepared by reaction of the parent acid compound with an amine, or basic groups reacted to form an acylated base derivative (e.g., a lower alkylamide).

The term “pharmaceutically acceptable salt,” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent (e.g., a compound of Formula I), an animal or a cell within an animal more susceptible, or more responsive, to the biological effects (e.g., promotion or retardation of an aspect of cellular function including, but not limited to, cell growth, proliferation, invasion, angiogenesis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least 10%, at least 20%, at least 30%, at least 40%, at least 50%, at least 60%, at least 70%, at least 80%, at least 90%, at least 100%, at least 150%, at least 200%, at least 350%, at least 300%, at least 350%, at least 400%, at least 450%, or at least 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, including for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis. It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “apoptosis modulating agents,” as used herein, refers to agents which are involved in modulating (e.g., inhibiting, decreasing, increasing, promoting) apoptosis. Examples of apoptosis modulating agents include proteins which comprise a death domain such as, but not limited to, Fas/CD95, TRAMP, TNF RI, DR1, DR2, DR3, DR4, DR5, DR6, FADD, and RIP. Other examples of apoptotic modulating agents include, but are not limited to, TNFα, Fas ligand, antibodies to Fas/CD95 and other TNF family receptors, TRAIL, antibodies to TRAIL-R1 or TRAIL-R2, Bcl-2, p53, BAX, BAD, Akt, CAD, P13 kinase, PP1, and caspase proteins. Modulating agents broadly include agonists and antagonists of TNF family receptors and TNF family ligands. Apoptosis modulating agents may be soluble or membrane bound (e.g. ligand or receptor). Preferred apoptosis modulating agents are inducers of apoptosis, such as TNF or a TNF-related ligand, particularly a TRAMP ligand, a Fas/CD95 ligand, a TNFR-1 ligand, or TRAIL.

The double biphenolic compound gossypol (compound 1) has been demonstrated to be a potent inhibitor of Bcl-2 and Bcl-xL and to have strong anti-cancer activity (Flack et al., J. Clin. Endocrinol. Metab. 76:1019 (1993); Bushunow et al., J. Neuro-Oncol. 43:79, (1999); Van Poznak et al., Breast Cancer Res. Treat. 66:239 (2001); U.S. Patent Application Nos. 2003/0008924; 2004/0214902). Based on NMR studies of the gossypol/Bcl-xL interaction followed by computational structure-based modeling, a series of isoflavone analogs, exemplified by compound 2, were designed, synthesized and shown to have anti-cancer activity. Based on these studies, a class of compounds that inhibit anti-apoptotic Bcl-2 family members has been identified.

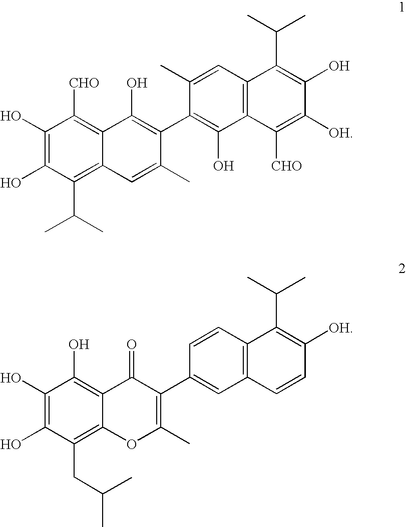

The inhibitors of anti-apoptotic Bcl-2 family members of the present invention are compounds having Formula I: 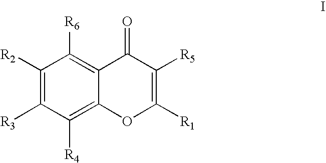
or a pharmaceutically acceptable salt or prodrug thereof, wherein:
R₁ is H, OH, F, Cl, Br, I, or optionally substituted alkyl, cycloalkyl, alkenyl, cycloalkenyl, alkynyl, aryl, heteroaryl, or heterocyclic;
   R₂, R₃, R₄, R₅, and R₆ are independently H, F, Cl, Br, I, OH, or optionally substituted alkyl, cycloalkyl, alkenyl, cycloalkenyl, alkynyl, aryl, heteroaryl, heterocyclic, CO₂R′, C(O)NR′R″, SO₂NR′R″, SR′, OR′, NR″C(O)R′, NR′SO₂R″, or NR′R″;
   R′ and R″ are independently H or optionally substituted alkyl, cycloalkyl, alkenyl, cycloalkenyl, alkynyl, aryl, heteroaryl, or heterocyclic, or R′ and R″ together with the N to which they are attached form a heterocyclic or heteroaryl ring.

In one embodiment, the compounds of Formula I have Formula II: 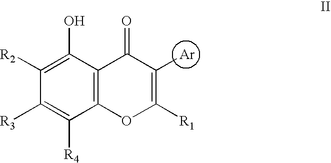
or a pharmaceutically acceptable salt or prodrug thereof, wherein:
Ar is optionally substituted aryl or heteroaryl.

In another embodiment, the compounds of Formula I have Formula III: 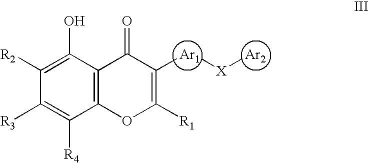
or a pharmaceutically acceptable salt or prodrug thereof, wherein:
Ar₁ and Ar₂ are independently optionally substituted aryl or heteroaryl;
X is O, NR′, SO₂, S, C(O)N(R′), SO₂NR′, R′NCO, R′NSO₂, N(R′)R″, N(R′)—R″—N(R′″), R′, OR′, OR′O, or C(O)N(R′)R″; and
R′, R″, and R′″ are independently H or optionally substituted alkyl, cycloalkyl, alkenyl, cycloalkenyl, alkynyl, aryl, heteroaryl, or heterocyclic, or two of R′, R″, and R′″ form a heterocyclic or heteroaryl ring.

In another embodiment, the compounds of Formula I have Formula IV: 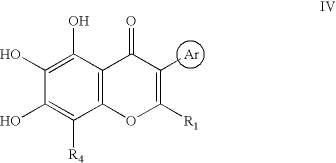
or a pharmaceutically acceptable salt or prodrug thereof.

In another embodiment, the compounds of Formula I have Formula V: 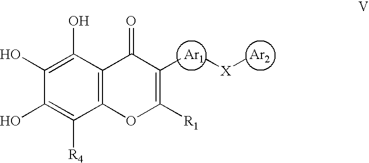
or a pharmaceutically acceptable salt or prodrug thereof.

In another embodiment, the compounds of Formula I have Formula VI: 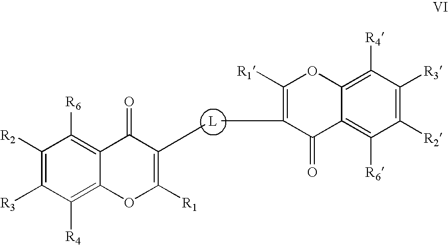
or a pharmaceutically acceptable salt or prodrug thereof; wherein
L is optionally substituted aryl, bi-aryl, heteroaryl, heterocyclic, alkyl, cycloalkyl, alkenyl, cycloalkenyl, alkynyl, ether, ester, amine, amide, sulfonyl, sulfonamide, or thioether;
R₁ and R₁′ are independently H, OH, F, Cl, Br, I, or optionally substituted alkyl, cycloalkyl, alkenyl, cycloalkenyl, alkynyl, aryl, heteroaryl, or heterocyclic; and R₂, R₂′, R₃, R₃′, R₄, R₄′, R₆, and R₆′ are independently H, F, Cl, Br, I, OH, or optionally substituted alkyl, cycloalkyl, alkenyl, cycloalkenyl, alkynyl, aryl, heteroaryl, heterocyclic, CO₂R′, C(O)NR′R″, SO₂NR′R″, SR′, OR′, NR″C(O)R′, NR′SO₂R″, or NR′R″.

Useful alkyl groups include straight-chained or branched C₁₋₁₈ alkyl groups, especially methyl, ethyl, propyl, isopropyl, t-butyl, sec-butyl, 3-pentyl, adamantyl, norbornyl, and 3-hexyl groups.

Useful alkenyl groups include straight-chained or branched C₂₋₁₈ alkyl groups, especially ethenyl, propenyl, isopropenyl, butenyl, isobutenyl, and hexenyl.

Useful alkynyl groups are C₂₋₁₈ alkynyl groups, especially ethynyl, propynyl, butynyl, and 2-butynyl groups

Useful cycloalkyl groups are C₃₋₈ cycloalkyl. Typical cycloalkyl groups include cyclopropyl, cyclobutyl, cyclopentyl, cyclohexyl and cycloheptyl.

Useful cycloalkenyl groups are C₃₋₈ cycloalkyl. Typical cycloalkenyl groups include cyclopropenyl, cyclobutenyl, cyclopentenyl, cyclohexenyl and cycloheptenyl.

Useful aryl groups include C₆₋₁₄ aryl, especially phenyl, naphthyl, phenanthrenyl, anthracenyl, indenyl, azulenyl, biphenyl, biphenylenyl, and fluorenyl groups.

Useful heteroaryl groups include thienyl, benzo[b]thienyl, naphtho[2,3-b]thienyl, thianthrenyl, furanyl, pyranyl, benzofuranyl, isobenzofuranyl, chromenyl, chromenonyl, xanthenyl, phenoxanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, 1,2-benzoisoxazol-3-yl, benzimidazolyl, 2-oxindolyl, and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide, pyrimidinyl N-oxide, and the like.

Useful heterocyclic groups include tetrahydrofuranyl, tetrahydropyranyl, tetrahydroquinolinyl, piperidinyl, piperizinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl, tetramoyl, or tetrahydroisoquinolinyl groups.

Optional substituents include one or more alkyl; halo; haloalkyl; cycloalkyl; hydroxy; aryl optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; aryloxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; amido optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; aralkyl; heteroaryl optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; heteroaryloxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; alkoxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; alkylthio; arylthio; amido; amino; acyloxy; arylacyloxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; diphenylphosphinyloxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; heterocyclo optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, heteroaryl, amino acid substituted sulfonyl, or amino acid derivative substituted sulfonyl groups; heterocycloalkoxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; partially unsaturated heterocycloalkyl optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups; or partially unsaturated heterocycloalkyloxy optionally substituted with one or more lower alkyl, lower alkoxy, methylenedioxy, halo, haloalkyl, hydroxy, acyl, aminosulfonyl, arylsulfonyl, aryl, aryloxy, acyloxy, amido, or heteroaryl groups. Additionally, more than one optional substituent may be linked together, e.g., amido linked to heterocyclo linked to aryl, etc.

Particular optional substituents include, without limitation, isopropyl, hydroxyl, methyl, ethoxy, ethyl, isobutyl, 2-methyl-5,6,7-methoxy-8-isobutyl-chromen-4-on-3-yl, 4-(2-methyl-5,6,7-hydroxy-8-isobutyl-chromen-4-on-3-yl)phenyl, N-benzamido, 2-methyl-5,6,7-hydroxy-8-isobutylchromen-4-on-3-yl, carboxymethyl, N-(3-isopropylphenyl)amido, carboxyl, N-(2-isopropylphenyl)amido, N-phenylamido, N-(1(S)-carboxynethylisopentyl)amido, N-(1-benzylpiperidin-4-yl)amido, N-[1 (S)-carboxymethyl-2-indol-2-yl)-ethyl]amido, N-(1-carboxymethylbenzyl)amido, N-[(2-indol-3-yl)-ethyl]amido, N-(diphenyl)amido, N-(1 (S)-carboxymethyl-2-phenylethyl)amido, phenyl, N-(adamantan-1-yl)amido, chloro, N-(naphth-2-yl)amido, N-[(1(S),2-dicarboxymethyl)ethyl]amido, [4-(3-methoxyphenyl)piperazin-1-yl]carbonyl, N-(2,2-diphenylethyl)amido, [(4-benzyl)[1,4]diazepan-1-yl]carbonyl, N-[1-benzyl-2-(4-methyl-piperazin-1-yl-2-oxo)ethyl]amido, N-[(1-benzyl-2-oxo-2-{4-[5-(2-oxo-hexahydro-thieno[3,4-d]imidazol-6-yl)-pentanoyl]-piperazin-1-yl}-ethyl]amido, N-[(1 (S)-carboxymethyl-2-phenyl)ethyl]amido, (4-phenylpiperazin-1-yl)carbonyl, and (4-benzylpiperidin-1-yl)carbonyl.

Certain of the compounds of the present invention may exist as stereoisomers including optical isomers. The invention includes all stereoisomers and both the racemic mixtures of such stereoisomers as well as the individual enantiomers that may be separated according to methods that are well known to those of skill in the art.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. It will be readily apparent to one of ordinary skill in the art that the compounds defined above can be synthesized by substitution of the appropriate reagents and agents in the syntheses shown below.

The synthesis of compounds having Formula I may be carried as exemplified in Scheme I for the synthesis of compound 2. 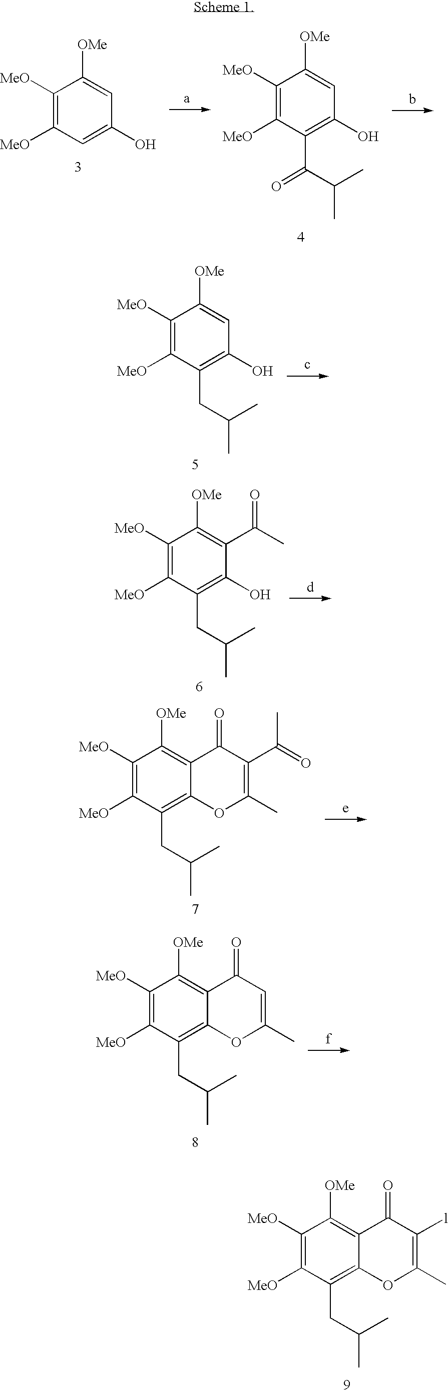 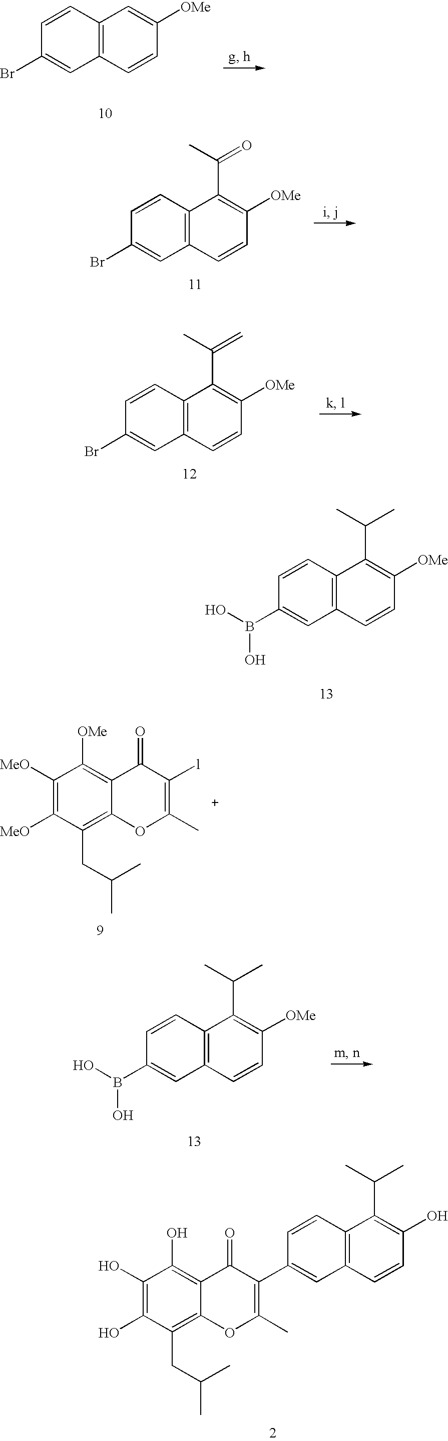
Reagents and conditions: (a) BF₃-Et₂O, isobutyryl chloride, 1,2-dichloroethane, reflux; (b) Et₃SiH, CF₃CO₂H; (c) BF₃-Et₂O, acetyl chloride, 1,2-dichloroethane, reflux; (d) AcONa, (AcO)₂O; (e) Na₂CO₃, H₂₀, 1,4-dioxane; (f) 12, CF₃CO₂Ag; (g) BF₃-Me₂S, (AcO)₂O, dichloromethane; (h) MDF, MeI, K₂CO₃; (i) THF, MeMgBr; (j) toluene, PTSA, reflux; (k) t-BuLi, B(OMe)₃, THF; (1) H₂, Pd—C; (m) Pd(dpf)₂Cl₂-CH₂Cl₂, Na₂CO₃, DMF, H₂O, EtOH; (n) CH₂Cl₂, BBr₃, 0° C.˜1 R.T.

Other compounds may be obtained as shown in Scheme 2 by treating compound 9 with different boronic acids and following the same procedure. 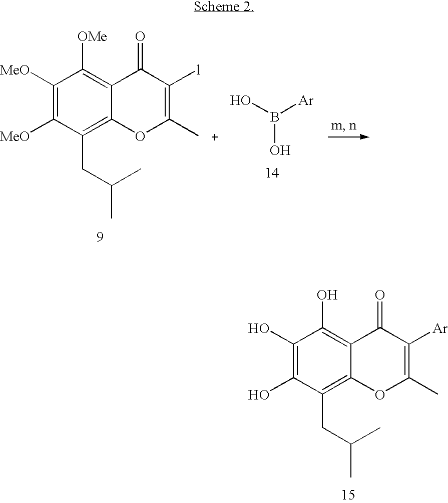
Reagents and conditions: (m) Pd(dpf)₂Cl₂—CH₂Cl₂, Na₂CO₃, DMF, H₂O, EtOH; (n) CH₂Cl₂, BBr₃, 0° C.˜1 R.T.

Scheme 3 shows an improved cyclization method to chromen-4-one ring formation and synthesis of compound 20. Briefly, α-acetophenol 6 is converted to propionate ester 16 by acylation in pyridine at room temperature. Compound 16 is treated with sodium hydride in anhydrous DMF at 0° C. to give 1,3-diketone intermediate. The reaction is quenched by the cautious addition of acetic acid, workup with ethyl acetate and water. The crude intermediate is used in the acid catalyzed intramolecular cyclization without further purification. Compound chromen-4-one 17 can be obtained from acetophenol 6 with a total yield of 86%. By following a similar procedure mentioned before, chromen-4-one 17 is converted to symmetrical compound 19 by iodization and palladium catalyzed Suzuki coupling. The polyhydroxyl chromen-4-one 20 is obtained by treating hexamethyl ether 19 with refluxing acetic acid and hydrobromic acid.

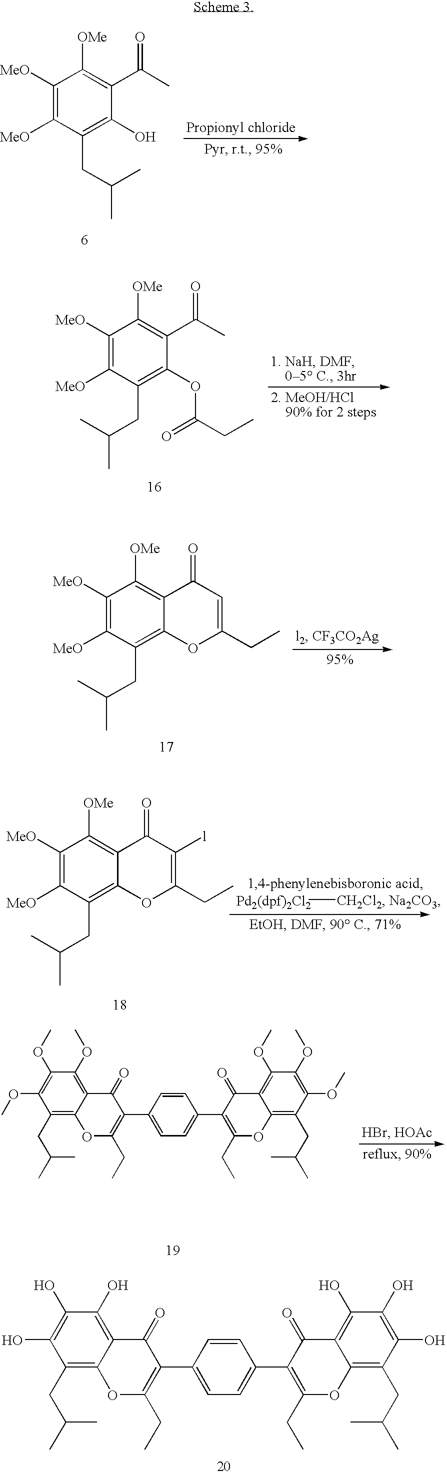

An important aspect of the present invention is that compounds of Formula I induce apoptosis and also potentiate the induction of apoptosis in response to apoptosis induction signals. Therefore, it is contemplated that these compounds sensitize cells to inducers of apoptosis, including cells that are resistant to such inducers. The anti-apoptotic Bcl-2 family member inhibitors of the present invention can be used to induce apoptosis in any disorder that can be treated, ameliorated, or prevented by the induction of apoptosis. Thus, the present invention provides compositions and methods for targeting animals characterized as overexpressing an anti-apoptotic Bcl-2 family member protein. In some of the embodiments, the cells (e.g., cancer cells) show elevated expression levels of anti-apoptotic Bcl-2 family member proteins as compared to non-pathological samples (e.g., non-cancerous cells). In other embodiments, the cells operationally manifest elevated expression levels of anti-apoptotic Bcl-2 family member proteins by virtue of executing the apoptosis program and dying in response to an inhibiting effective amount of a compound of Formula I, said response occurring, at least in part, due to the dependence in such cells on anti-apoptotic Bcl-2 family member protein function for their survival.

In another embodiment, the invention pertains to modulating an apoptosis associated state which is associated with one or more apoptosis modulating agents. Examples of apoptosis modulating agents include, but are not limited to, Fas/CD95, TRAMP, TNF RI, DR1, DR2, DR3, DR4, DR5, DR6, FADD, RIP, TNFα, Fas ligand, TRAIL, antibodies to TRAIL-R1 or TRAIL-R2, Bcl-2, p53, BAX, BAD, Akt, CAD, P13 kinase, PP1, and caspase proteins. Other agents involved in the initiation, decision and degradation phase of apoptosis are also included. Examples of apoptosis modulating agents include agents, the activity, presence, or change in concentration of which, can modulate apoptosis in a subject. Preferred apoptosis modulating agents are inducers of apoptosis, such as TNF or a TNF-related ligand, particularly a TRAMP ligand, a Fas/CD95 ligand, a TNFR-1 ligand, or TRAIL.

In some embodiments, the compositions and methods of the present invention are used to treat diseased cells, tissues, organs, or pathological conditions and/or disease states in an animal (e.g., a mammalian subject including, but not limited to, humans and veterinary animals). In this regard, various diseases and pathologies are amenable to treatment or prophylaxis using the present methods and compositions. A non-limiting exemplary list of these diseases and conditions includes, but is not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic. In other embodiments, the cancer cells being treated are resistant to anticancer agents.

In some embodiments, infections suitable for treatment with the compositions and methods of the present invention include, but are not limited to, infections caused by viruses, bacteria, fungi, mycoplasma, prions, and the like.

Some embodiments of the present invention provide methods for administering an effective amount of a compound of Formula I and at least one additional therapeutic agent (including, but not limited to, chemotherapeutic antineoplastics, apoptosis modulating agents, antimicrobials, antivirals, antifungals, and anti-inflammatory agents) and/or therapeutic technique (e.g., surgical intervention, and/or radiotherapies).

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In preferred embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of Formula I and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., Emeryville, CA |
| (des-alanyl-1, serine-125 human interleukin-2) | | |
| Alemtuzumab | Campath | Millennium and ILEX |
| (IgG1κ anti CD52 antibody) | | Partners, LP, Cambridge, MA |
| Alitretinoin | Panretin | Ligand Pharmaceuticals, Inc., |
| (9-cis-retinoic acid) | | San Diego CA |
| Allopurinol | Zyloprim | GlaxoSmithKline, Research |
| (1,5-dihydro-4 H-pyrazolo[3,4-d]pyrimidin-4- | | Triangle Park, NC |
| one monosodium salt) | | |
| Altretamine | Hexalen | US Bioscience, West |
| (N,N,N′,N′,N″,N″,-hexamethyl-1,3,5-triazine-2, | | Conshohocken, PA |
| 4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca Pharmaceuticals, |
| (1,3-Benzenediacetonitrile, a,a,a′,a′- | | LP, Wilmington, DE |
| tetramethyl-5-(1H-1,2,4-triazol-1-ylmethyl)) | | |
| Arsenic trioxide | Trisenox | Cell Therapeutic, Inc., Seattle, |
| | | WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain | | Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8-pentamethyl- | | |
| 2-napthalenyl) ethenyl] benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics produced by | | NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2−)-0,0′]-,(SP-4-2)) | | |
| Carmustine | BCNU, | Bristol-Myers Squibb |
| (1,3-bis(2-chloroethyl)-1-nitrosourea) | BiCNU | |
| Carmustine with Polifeprosan 20 Implant | Gliadel | Guilford Pharmaceuticals, |
| | Wafer | Inc., Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, 2- | R. W. Johnson |
| (2-chloro-2′-deoxy-b-D-adenosine) | CdA | Pharmaceutical |
| | | Research Institute, Raritan, NJ |
| Cyclophosphamide | Cytoxan, | Bristol-Myers Squibb |
| (2-[bis(2-chloroethyl)amino] tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC-Dome | Bayer AG, Leverkusen, |
| (5-(3,3-dimethyl-1-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand Oaks, |
| (recombinant peptide) | | CA |
| daunorubicin liposomal | DanuoXome | Nexstar Pharmaceuticals, Inc., |
| ((8S-cis)-8-acetyl-10-[(3-amino-2,3,6-trideoxy- | | Boulder, CO |
| a-L-lyxo-hexopyranosyl)oxy]-7,8,9,10- | | |
| tetrahydro-6,8,11-trihydroxy-1-methoxy-5,12- | | |
| naphthacenedione hydrochloride) | | |
| Daunorubicin HCl, daunomycin | Cerubidine | Wyeth Ayerst, Madison, NJ |
| ((1S,3S)-3-Acetyl-1,2,3,4,6,11-hexahydro- | | |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., Hopkinton, MA |
| (recombinant peptide) | | |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, Inc., |
| ((2R,3S)-N-carboxy-3-phenylisoserine, N-tert- | | Bridgewater, NJ |
| butyl ester, 13-ester with 5b-20-epoxy- | | |
| 12a,4,7b,10b,13a-hexahydroxytax-11-en-9-one | | |
| 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin | Pharmacia & Upjohn |
| | PFS | Company |
| | Intravenous | |
| | injection | |
| doxorubicin liposomal | Doxil | Sequus Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo Alto, CA |
| | injection | |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral | | Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5-(10)- | | |
| triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca Pharmaceuticals |
| (acetate salt of [D-Ser(But)⁶,Azgly¹⁰]LHRH; | Implant | |
| pyro-Glu-His-Trp-Ser-Tyr-D-Ser(But)-Leu- | | |
| Arg-Pro-Azgly-NH2 acetate | | |
| [C₅₉H₈₄N₁₈O₁₄.(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperidinopiperidino) | | Company |
| carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., Seattle, WA |
| (L-Glutamic acid, N[4[[(2amino-5-formyl- | Leucovorin | |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research Foundation, |
| ((−)-(S)-2,3,5,6-tetrahydro-6-phenylimidazo | | Titusville, NJ |
| [2,1-b] thiazole monohydrochloride | | |
| C₁₁H₁₂N₂S.HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way Exton, |
| (9-methoxy-7H-furo[3,2-g][1]-benzopyran-7- | | Pa |
| one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin- | Organon, Inc., West Orange, |
| | 50 | NJ |
| Nofetumomab | Verluma | Boehringer Ingelheim Pharma |
| | | KG, Germany |
| Oprelvekin | Neumega | Genetics Institute, Inc., |
| (IL-11) | | Alexandria, VA |
| Oxaliplatin | Eloxatin | Sanofi Synthelabo, Inc., NY, |
| (cis-[(1R,2R)-1,2-cyclohexanediamine-N,N′] | | NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R,3S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, Inc., |
| ((monomethoxypolyethylene glycol | (Pegademase | Bridgewater, NJ |
| succinimidyl) 11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis Pharmaceutical |
| | | Co., Rockville, MD |
| Pipobroman | Vercyte | Abbott Laboratories, Abbott |
| | | Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, Inc., |
| | | Vancouver, |
| | | Canada |
| Procarbazine | Matulane | Sigma Tau Pharmaceuticals |
| (N-isopropyl-μ-(2-methylhydrazino)-p- | | Inc., Gaithersburg, MD |
| toluamide monohydrochloride) | | |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and | | |
| b)-D-glucopyranose and 220 mg citric acid | | |
| anhydrous) | | |
| Talc | Sclerosol | Bryan, Corp., Woburn, MA |
| (Mg₃Si₄O₁₀(OH)₂) | | |
| Tamoxifen | Nolvadex | AstraZeneca Pharmaceuticals |
| ((Z)2-[4-(1,2-diphenyl-1-butenyl) phenoxy]-N, | | |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine, 1,1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′: 6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, WA |
| (recombinant murine immunotherapeutic | | |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀.H₂SO₄) | | |
| Vincristine | Oncovin | Eli Lilly |
| (C₄₆H₅₆N₄O₁₀.H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity but are not currently approved by the U.S. Food and Drug Administration or other counterpart agencies or are undergoing evaluation for new uses. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifamib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

Preferred conventional anticancer agents for use in administration with the present compounds include, but are not limited to, adriamycin, 5-fluorouracil, etoposide, camptothecin, actinomycin D, mitomycin C, cisplatin, docetaxel, gemcitabine, carboplatin, oxaliplatin, bortezomib, gefitinib, and bevacizumab. These agents can be prepared and used singularly, in combined therapeutic compositions, in kits, or in combination with immunotherapeutic agents, and the like.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present invention provides methods for administering a compound of Formula I with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. The dose of radiation is preferably fractionated for maximal target cell exposure and reduced toxicity.

The total dose of radiation administered to an animal preferably is about 0.01 Gray (Gy) to about 100 Gy. More preferably, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce destruction of the targeted cells. If stretched over a period, radiation preferably is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. Preferably, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of Formula I and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. Preferably, about 0.01 to about 10 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, and most preferably, from about 0.01 to about 5 mg/kg.

The unit oral dose may comprise from about 0.01 to about 1000 mg, preferably about 0.1 to about 100 mg of the compound. The unit dose may be administered one or more times daily as one or more tablets or capsules each containing from about 0.1 to about 100, conveniently about 0.25 to 50 mg of the compound or its solvates.

In a topical formulation, the compound may be present at a concentration of about 0.01 to 100 mg per gram of carrier. In a preferred embodiment, the compound is present at a concentration of about 0.07-1.0 mg/ml, more preferably, about 0.1-0.5 mg/ml, most preferably, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. Preferably, the preparations, particularly those preparations which can be administered orally or topically and which can be used for the preferred type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by injection, topically or orally, contain from about 0.01 to 99 percent, preferably from about 0.25 to 75 percent of active compound(s), together with the excipient.

The pharmaceutical compositions of the invention may be administered to any animal which may experience the beneficial effects of the compounds of the invention. Foremost among such animals are mammals, e.g., humans, although the invention is not intended to be so limited. Other animals include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### 1-(6-Hydroxy-2,3,4-trimethoxy-phenyl)-2-methyl-propan-1-one

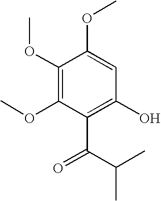

To a solution of 3,4,5-trimethoxyphenol (9.21 g, 50 mmol) in 150 mL 2,2-dichloroethane, boron trifluoride diethyl etherate (28.5 mL, 220 mmol) and isobutyryl chloride (5.9 mL, 55 mmol) were added. The resulting mixture was refluxed for 12 hours, and the solvent was removed in vacuo. To the resulting residue, 80 mL 3 M HCl was added under ice bath and the mixture was stirred for 1 hour at room temperature, then extracted with ethyl acetate, dried over Na₂SO₄, purified by silica gel column chromatography (hexane:ethyl acetate=6:1), and product was obtained. Yield: 80%.

¹H NMR (CDCl₃, 300 MHz), δ 13.45 (s, 1H); 6.26 (s, 1H); 4.01 (s, 3H); 3.94 (s, 3H); 3.87 (s, 3H); 3.80˜3.70 (m, 1H); 1.21 (d, J=6.76 Hz, 6H); ¹³C NMR (CDCl₃, 75 MHz), δ 162.00; 159.64; 154.88; 134.63; 107.35; 96.20; 61.54; 60.94; 56.01; 39.03; 19.46.

### EXAMPLE 2

### 2-Isobutyl-3,4,5-trimethoxy-phenol

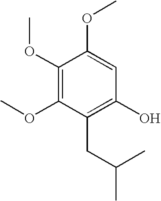

1-(6-Hydroxy-2,3,4-trimethoxy-phenyl)-2-methyl-propan-1-one (5.1 g, 20 mmol) was dissolved in 30 mL trifluoride acetic acid and 3 mL triethylsilane was added at room temperature. The resulting solution was stirred overnight, and the solvent was removed in vacuo. The residue was purified by silica gel column chromatography (hexane:ethyl acetate=4:1), and product was obtained. Yield: >95%.

¹H NMR (CDCl₃, 300 MHz), δ 6.27 (s, 1H); 3.90 (s, 3H); 3.85 (s, 3H); 3.82 (s, 3H); 2.43 (d, J=7.35 Hz, 2H); 1.91˜1.80 (m, 1H); 0.89 (d, J=6.63 Hz, 6H).

### EXAMPLE 3

### 1-(2-Hydroxy-3-isobutyl-4,5,6-trimethoxy-phenyl)-ethanone

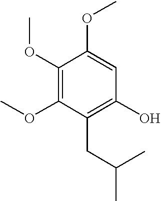

To a solution of the compound of Example 2 (4.86 g, 20 mmol) in 80 mL 2,2-dichloroethane, boron trifluoride diethyl etherate (14.3 mL, 110 mmol) and acetyl chloride (1.75 mL, 22 mmol) were added. The resulting mixture was refluxed for 12 hours, and the solvent was removed in vacuo. To the resulting residue, 50 mL 3 M HCl was added under ice bath and the mixture was stirred for 1 hour at room temperature, then extracted with ethyl acetate, dried over Na₂SO₄, purified by silica gel column chromatography (hexane:ethyl acetate=8:1), and compound were obtained. Yield: 65%.

¹H NMR (CDCl₃, 300 MHz), δ 13.28 (s, 1H); 3.99 (s, 3H); 3.96 (s, 3H); 3.87 (s, 3H); 2.69 (s, 3H); 2.49 (d, J=7.27 Hz, 2H); 1.97˜1.88 (m, 1H); 0.92 (d, J=6.65 Hz, 6H); ¹³C NMR (CDCl₃, 75 MHz), δ 204.05; 158.99; 153.84; 138.04; 118.47; 110.46; 61.00; 60.82; 60.64; 32.25; 31.99; 28.20; 22.62.

### EXAMPLE 4

### 3-Acetyl-8-isobutyl-5,6,7-trimethoxy-2-methyl-chromen-4-one

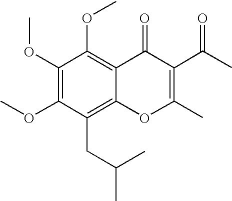

To a solution of the compound of Example 3 (5.65 g, 20 mmol) in 60 mL acetic anhydride, sodium acetate (4.1 g, 50 mmol) was added. The resulting mixture was refluxed for 12 hours, and the solvent was removed in vacuo. The residue was dissolved in 100 mL water and was extracted with ethyl acetate, dried over Na₂SO₄, purified by silica gel column chromatography (hexane:ethyl acetate=8:1), and compound was obtained. Yield: 87%.

¹H NMR (CDCl₃, 300 MHz), δ 3.92 (s, 3H); 3.91 (s, 3H); 3.82 (s, 3H); 2.50 (s, 3H); 2.34 (d, J=7.20 Hz, 2H); 2.23 (s, 3H); 1.86˜1.75 (m, 1H); 0.89 (d, J=6.64 Hz, 6H); ¹³C NMR (CDCl₃, 75 MHz), δ 200.25; 171.08; 169.37; 166.34; 154.25; 149.77; 143.71; 141.08; 124.32; 124.23; 61.66; 60.84; 60.61; 33.49; 31.63; 28.82; 22.64; 20.74.

### EXAMPLE 5

### 8-Isobutyl-5,6,7-trimethoxy-2-methyl-chromen-4-one

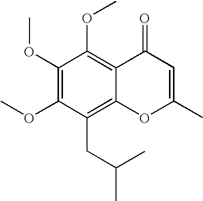

To a solution of the compound of Example 4 (3.50 g, 10.1 mmol) in 30 mL 1,4-dioxane, 30 mL water and sodium carbonate (2.12 g, 20 mmol) were added. The resulting mixture was refluxed for 1.5 hours and extracted with ethyl acetate, dried over Na₂SO₄, purified by silica gel column chromatography (hexane:ethyl acetate=1:1), and compound was obtained. Yield: 60%.

¹H NMR (CDCl₃, 300 MHz), δ 6.03 (s, 1H); 4.05 (s, 3H); 3.95 (s, 3H); 3.93 (s, 3H); 2.68 (d, J=7.20 Hz, 2H); 2.32 (s, 3H); 1.97˜1.88 (m, 1H); 0.95 (d, J=6.66 Hz, 6H).

### EXAMPLE 6

### 3-Iodo-8-isobutyl-5,6,7-trimethoxy-2-methyl-chromen-4-one

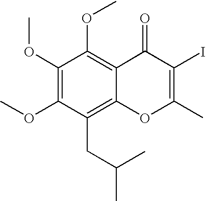

To a solution of the compound of Example 5 (4.58 g, 15.0 mmol) in 80 mL dichloromethane, iodine (5.08 g, 20 mmol) and silver trifluoroacetate (4.42 g, 20 mmol) were added at 0° C. The resulting mixture was stirred overnight. The solid was removed by filtration, and the solvent was removed in vacuo. The residue was purified by silica gel column chromatography (hexane:ethyl acetate=8:1), and compound was obtained. Yield 96%.

¹H NMR (CDCl₃, 300 MHz), δ 4.07 (s, 3H); 3.96 (s, 3H); 3.88 (s, 3H); 2.73 (s, 3H); 2.68 (d, J=7.20 Hz, 2H); 1.99˜1.89 (m, 1H); 0.94 (d, J=6.66 Hz, 6H); ¹³C NMR (CDCl₃, 75 MHz), δ 172.44; 164.11; 156.39; 151.25; 150.71; 144.11; 118.96; 111.42; 89.78; 62.14; 61.27; 61.22; 32.55; 28.96; 25.22; 22.65.

### EXAMPLE 7

### 6-Bromo-1-acetyl-2-methoxy-naphthalene

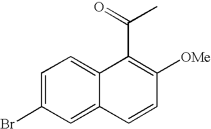

A solution of 10 mmol of acetic anhydride in 2 mL CH₂Cl₂ was added by syringe to a vigorously stirred solution of 10 mmol of Me₂S:BF₃ complex in 10 mL CH₂Cl₂ at −78° C. under argon. The mixture was stirred for 10 minutes, then a solution of 5 mmol 6-bromo-2-methoxy-naphthalene in 3 mL CH₂Cl₂ was added and the resulting solution was stirred at −78° C. for an additional 15 minutes. The solution was allowed to warm to room temperature, and stirred for 24 hours, then poured into a mixture of saturated NaHCO₃, extracted with CH₂Cl₂, and dried over Na₂SO₄. The solvent was removed in vacuo and the residue was dissolved in mL DMF. To the resulting solution 10 mmol Na₂CO₃ and 2 mL MeI were added. The resulting mixture was stirred overnight at room temperature. The solid was removed by filtration, and the solvent was removed in vacuo. The residue was purified by silica gel column chromatography (hexane:ethyl acetate=4:1), and compound was obtained. Yield: 85%.

¹H NMR (CDCl₃, 300 MHz), δ 7.97 (s, 1H); 7.82 (d, J=8.27 Hz, 1H); 7.68 (d, J=7.80 Hz, 1H); 7.55 (d, J=8.59 Hz, 1H); 7.32 (d, J=9.00 Hz, 1H); 4.00 (s, 3H); 2.66 (s, 3H).

### EXAMPLE 8

### 6-Bromo-1-isopropenyl-2-methoxy-naphthalene

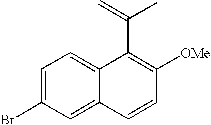

To a solution of the compound of Example 7 (2.78 g, 10 mmol) in 50 mL THF, 11 mL MeMgCl (1.0 M, 11 mmol) was added at −78° C. The solution was stirred for 2 hours, and the reaction was quenched by 10 mL saturated NH₄Cl, extracted with ethyl acetate, and dried over Na₂SO₄. The solvent was removed in vacuo and the residue was dissolved in 25 mL toluene. To the solution 100 mg PTSA was added and was refluxed for 2.0 hours. The solvent was removed in vacuo and the residue was purified by silica gel column chromatography (hexane:ethyl acetate=7:1) and compound was obtained. Yield: 60%.

¹H NMR (CDCl₃, 300 MHz), δ 7.95 (d, J=1.99 Hz, 1H); 7.86 (d, J=9.06 Hz, 1H); 7.72 (d, J=9.02 Hz, 1H); 7.50 (dd, J=2.06, 9.05 Hz, 1H); 7.32 (d, J=9.08 Hz, 1H); 5.55 (s, 1H); 4.96 (s, 1H); 3.97 (s, 3H); 2.13 (s, 3H).

### EXAMPLE 9

### 1-Isopropenyl-2-methoxy-naphthyl-6-boronic acid

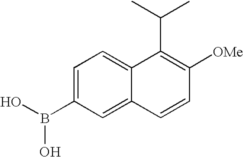

The boronic acid was synthesized under the standard procedure followed by hydrogenation.

General Synthetic Route to Isoflavone Analogues

To a dry flask, the compound of Example 6 (1.0 mmol), aromatic boronic acid (1.1 mmol), Na₂CO₃ (1.2 mmol) and [1,1′-bis(diphenylphosphino)ferrocene]dichloropalladium(Π) complex with dichloromethane (1:1) (30 mg) were added. 5 mL DMF, 2 mL EtOH and 2 mL H₂O were added by syringes. The resulting mixture was stirred at 60° C. under argon for 4˜8 hours. The solid was removed by filtration, and the solvent was removed in vacuo. The residue was purified by silica gel column chromatography (hexane:ethyl acetate=2:1˜4:1), and the coupling products were obtained (70%˜90%).

The coupling product (1.0 mmol) was dissolved in 25 mL CH₂Cl₂, and 3.3 mL BBr₃ (1.0 M in CH₂Cl₂) was added at −78° C. The mixture was allowed to warm to room temperature over 3.0 hours. MeOH (1.0 mL) was added to quench the reaction and the solvent was removed in vacuo. The residue was purified by silica gel column chromatography or crystallization from acetone-H₂O, and the isoflavones were obtained (60%˜75%).

### EXAMPLE 10

### 5,6,7-Trihydroxy-3-(6-hydroxy-5-isopropyl-naphthalen-2-yl)-8-isobutyl-2-methyl-chromen-4-one

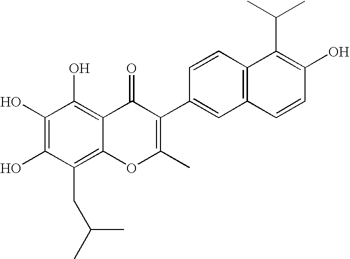

¹H NMR (CDCl₃, 300 MHz), δ 12.85 (s, 1H); 8.21 (d, J=8.91 Hz, 1H); 7.69 (s, 1H); 7.59 (d, J=8.66 Hz, 1H); 7.41 (d, J=8.84 Hz, 1H); 6.99 (d, J=8.62 Hz, 1H); 6.16 (s, 1H); 5.47 (s, 1H); 5.04 (s, 1H); 3.99˜3.83 (m, 1H); 2.73 (d, J=7.29 Hz, 2H); 2.38 (s, 3H); 2.09˜2.00 (m, 1H); 1.55 (d, J=7.03 Hz, 6H); 0.99 (d, J=6.64 Hz, 6H); ¹³C NMR (CDCl₃, 75 MHz), δ 181.86; 164.35; 148.93; 148.86; 143.81; 132.46; 130.45; 128.20; 127.86; 126.84; 126.45; 125.37; 120.83; 119.03; 106.34; 104.37; 34.66; 31.39; 28.66; 22.61; 21.09; 19.59; HRMS(EI, [M+H]⁺) Calcd: 449.1964. Found: 449.1973. Anal. Calcd for C₂₇H₂₈O₆-0.2H₂O: C, 71.73; H, 6.33. Found: C, 71.70; H, 6.32.

### EXAMPLE 11

### 5,6,7-Trihydroxy-3-(6-hydroxy-5-methyl-naphthalen-2-yl)-8-isobutyl-2-methyl-chromen-4-one

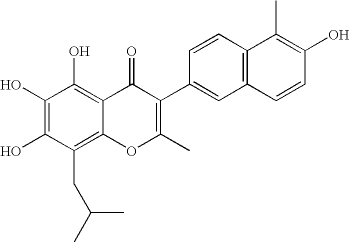

¹H NMR (DMSO-d₆, 300 MHz), δ 12.88 (s, 1H); 9.83 (s, 1H); 9.58 (s, 1H); 9.10 (s, 1H); 7.90 (d, J=8.76 Hz, 1H); 7.72 (s, 1H); 7.63 (d, J=8.94 Hz, 1H); 7.39 (d, J=8.67 Hz, 1H); 7.19 (d, J=8.94 Hz, 1H); 2.62 (d, J=7.11 Hz, 2H); 2.45 (s, 3H); 2.31 (s, 3H); 1.99˜1.90 (m, 1H); 0.92 (d, J=6.59 Hz, 6H); ¹³C NMR (DMSO-d₆, 75 MHz), δ 181.91; 164.90; 153.44; 152.79; 148.89; 146.04; 133.92; 130.96; 129.54; 129.14; 128.65; 127.77; 126.93; 123.46; 120.71; 119.13; 115.51; 106.48; 104.00; 32.17; 31.65; 18.99; 23.41; 20.29; HRMS(EI, [M+H]⁺) Calcd: 421.1651. Found: 421.1644. Anal. Calcd for C₂₅H₂₄O₆: C, 71.41; H, 5.75. Found: C, 71.14; H, 6.03.

### EXAMPLE 12

### 5,6,7-Trihydroxy-3-(6-hydroxy-naphthalen-2-yl)-8-isobutyl-2-methyl-chromen-4-one

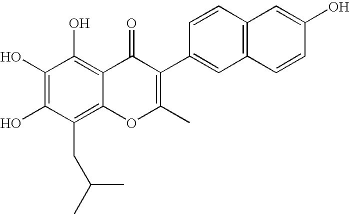

¹H NMR (DMSO-d₆, 300 MHz), δ 12.86 (s, 1H); 9.85 (s, 1H); 9.35 (s, 1H); 9.12 (s, 1H); 7.79˜7.71 (m, 2H); 7.51 (d, J=8.60 Hz, 1H); 7.33 (dd, J=1.46, 8.18 Hz, 1H); 7.16˜7.12 (m, 1H); 7.01˜6.95 (m, 1H); 2.61 (d, J=8.37 Hz, 2H); 2.31 (s, 3H); 1.99˜1.90 (m, 1H); 0.91 (d, J=6.62 Hz, 6H); ¹³C NMR (DMSO-d₆, 75 MHz), δ 181.93; 164.93; 156.61; 152.81; 148.91; 146.06; 134.83; 130.40; 129.78; 129.16; 128.41; 127.40; 126.58; 120.80; 119.74; 109.48; 106.51; 104.02; 32.18; 29.02; 23.43; 20.30; HRMS(EI, [M+H]⁺) Calcd: 407.1495. Found: 407.1497. Anal. Calcd for C₂₄H₂₂O₆: C, 70.92; H, 5.46. Found: C, 70.96; H, 5.61.

### EXAMPLE 13

### 5,6,7-Trihydroxy-8-isobutyl-2-methyl-3-naphthalen-2-yl-chromen-4-one

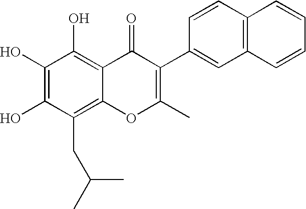

¹H NMR (CDCl₃, 300 MHz), δ 12.83 (s, 1H); 8.17˜7.80 (m, 3H); 7.56˜7.42 (m, 2H); 6.19 (s, 1H); 5.58 (s, 1H); 2.73 (d, J=7.18 Hz, 2H); 2.38 (s, 3H); 2.09˜2.00 (m, 1H); 0.99 (d, J=6.63 Hz, 6H); ¹³C NMR (CDCl₃, 75 MHz), δ 181.69; 164.31; 148.95; 148.90; 143.83; 133.32; 132.89; 129.63; 128.14; 128.08; 128.01; 127.72; 126.88; 126.33; 126.17; 120.95; 106.36; 104.34; 31.39; 28.65; 22.56; 19.57; HRMS(EI, [M+H]⁺) Calcd: 391.1545. Found: 391.1537. Anal. Calcd for C₂₃H₂₀O₅: C, 73.39; H, 5.36. Found: C, 73.53; H, 5.64.

### EXAMPLE 14

### 3-(6-Hydroxy-naphthalen-2-yl)-8-isobutyl-5,6,7-trimethoxy-2-methyl-chromen-4-one

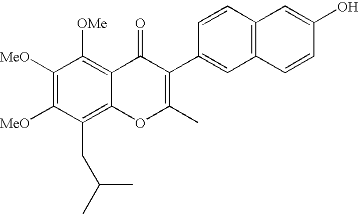

¹H NMR (CDCl₃, 300 MHz), δ 7.55 (s, 1H); 7.42˜7.34 (m, 2H); 7.23 (d, J=9.21 Hz, 1H); 7.03 (br, 1H): 6.88 (d, J=7.11 Hz, 1H): 6.80 (s, 1H); 4.05 (s, 3H); 4.01 (s, 3H); 3.97 (s, 3H); 2.75 (d, J=7.22 Hz, 2H); 2.30 (s, 3H); 2.05˜1.96 (m, 1H); 1.00 (d, J=6.62 Hz, 6H); ¹³C NMR (CDCl₃, 75 MHz), δ 177.70; 162.23; 156.38; 154.19; 151.82; 151.26; 143.93; 133.81; 129.22; 128.94; 128.41; 128.17; 127.36; 126.53; 123.83; 119.29; 118.32; 114.57; 109.46; 62.13; 61.32; 61.29; 32.49; 29.03; 22.74; 19.24; HRMS(EI, [M+H]⁺) Calcd: 449.1964. Found: 449.1965. Anal. Calcd for C₂₇H₂₈O₆: C, 72.30; H, 6.29. Found: C, 72.12; H, 6.11.

### EXAMPLE 15

### 3-(6-Ethoxy-naphthalen-2-yl)-8-isobutyl-5,6,7-trimethoxy-2-methyl-chromen-4-one

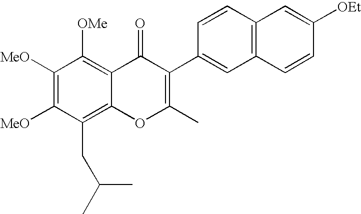

¹H NMR (CDCl₃, 300 MHz), δ 7.78 (d, J=8.48 Hz, 1H); 7.74 (d, J=9.78 Hz, 1H); 7.70 (s, 1H); 7.39 (dd, J=1.67, 8.40 Hz, 1H); 7.17 (s, 1H); 7.15 (dd, J=2.20, 7.00 Hz, 1H); 4.19 (q, J=6.68 Hz, 2H); 4.03 (s, 3H); 3.95 (s, 3H); 3.94 (s, 3H); 2.74 (dd, J=7.20 Hz, 2H); 2.32 (s, 3H); 2.05˜1.96 (m, 1H); 1.51 (t, J=6.97 Hz, 3H); 0.99 (d, J=6.65 Hz, 6H); ¹³C NMR (CDCl₃, 75 MHz), δ 176.43; 161.17; 157.11; 156.03; 151.66; 151.23; 143.75; 134.01; 129.46; 129.42; 128.97; 128.77; 128.38; 126.59; 123.40; 119.19; 118.96; 114.64; 106.36; 63.43; 62.06; 61.29; 61.23; 32.47; 29.03; 22.74; 19.17; 14.80; HRMS(EI, [M+H]⁺) Calcd: 477.2277. Found: 477.2273. Anal. Calcd for C₂₉H₃₂O₆: C, 73.09; H, 6.77. Found: C, 73.26; H, 7.13.

### EXAMPLE 16

### 5,6,7-Trihydroxy-8-isobutyl-2-methyl-3-phenyl-chromen-4-one

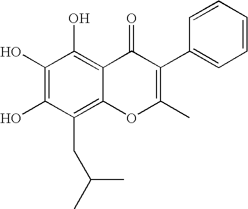

¹H NMR (CDCl₃, 300 MHz), δ 12.83 (s, 1H); 7.51˜7.28 (m, 5H); 6.15; (s, 1H); 5.49 (s, 1H); 2.71 (d, J=7.23 Hz, 2H); 2.34 (s, 3H); 2.05˜2.01 (m, 1H); 0.98 (d, J=6.66 Hz, 6H); ¹³C NMR (CDCl₃, 75 MHz), δ 181.58; 164.07; 148.87; 143.81; 132.07; 130.41; 128.54; 128.00; 126.83; 120.98; 110.59; 106.30; 104.32; 31.37; 28.63; 22.55; 19.49; HRMS(EI, [M+H]⁺) Calcd: 341.1389. Found: 341.1384. Anal. Calcd for C₂₀H₂₀O₅: C, 70.57; H, 5.92. Found: C, 70.37; H, 6.16.

### EXAMPLE 17

### 5,6,7-Trihydroxy-8-isobutyl-2-methyl-chromen-4-one

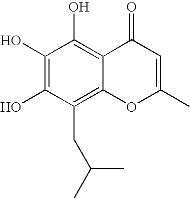

¹H NMR (DMSO-d₆, 300 MHz), δ 12.77 (s, 1H); 9.78 (s, 1H); 9.07 (s, 1H); 6.13 (s, 1H); 2.55 (d, J=7.20 Hz, 2H); 2.38 (s, 3H); 1.93˜1.84 (m, 1H); 0.86 (d, J=6.65 Hz, 6H); ¹³C NMR (DMSO-d₆, 75 MHz), δ 183.39; 168.10; 152.67; 149.43; 145.90; 129.14; 107.95; 106.76; 104.06; 32.17; 28.93; 23.35; 20.93; HRMS(EI, [M+H]⁺) Calcd: 265.1076. Found: 265.1077; Anal. Calcd for C₁₄H₁₆O₅: C, 63.63; H, 6.10. Found: C, 63.43; H, 6.23.

### EXAMPLE 18

### 3-Benzo[b]thiophen-2-yl-5,6,7-trihydroxy-8-isobutyl-2-methyl-chromen-4-one

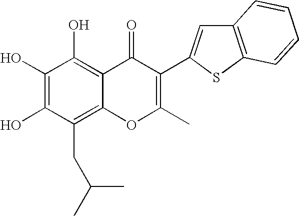

¹H NMR (CDCl₃, 300 MHz), δ 12.63 (s, 1H); 7.90˜7.81 (m, 2H); 7.42˜7.37 (m, 2H); 7.30 (s, 1H); 6.18 (s, 1H); 5.48 (s, 1H); 2.71 (d, J=7.24 Hz, 2H); 2.53 (s, 3H); 2.08˜1.08 (m, 1H); 0.99 (d, J=6.62 Hz, 6H); ¹³C NMR (CDCl₃, 75 MHz), δ 180.97; 165.83; 148.92; 148.60; 143.76; 140.94; 139.46; 133.11; 127.14; 125.84; 124.49; 124.25; 123.69; 122.11; 114.66; 106.56; 31.37; 28.63; 22.54; 20.03.

### EXAMPLE 19

### 3-(5-Ethyl-6-hydroxy-naphthalen-2-yl)-5,6,7-trihydroxy-8-isobutyl-2-methyl-chromen-4-one

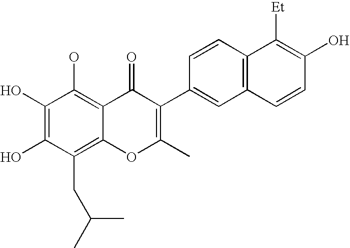

¹H NMR (CDCl₃, 300 MHz), δ 12.84 (s, 1H); 8.03 (d, J=8.73 Hz, 1H); 7.70 (d, J=1.68 Hz, 1H); 7.62 (d, J=8.97 Hz, 1H); 7.43 (dd, J=1.77; 8.73 Hz, 1H); 7.08 (d, J=8.97 Hz, 1H); 6.14 (s, 1H); 5.40 (s, 1H); 5.02 (s, 1H); 3.09 (q, J=7.35 Hz, 2H); 2.73 (d, J=7.27 Hz, 2H); 2.38 (s, 3H); 2.08˜2.00 (m, 1H); 1.32 (t, J=7.54 Hz, 3H); 0.99 (d, J=6.65 Hz, 6H).

### EXAMPLE 20

### 5,6,7-Trihydroxy-8-isobutyl-2-methyl-3-quinolin-3-yl-chromen-4-one

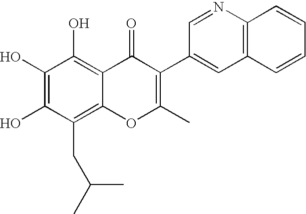

¹H NMR (DMSO-d₆, 300 MHz), δ 12.46 (br 1H); 10.03 (br, 1H); 9.70 (s, 1H); 9.18 (s, 1H); 8.30 (d, J=9.33 Hz, 1H); 8.27 (d, J=9.56 Hz, 1H); 8.11 (dd, J=7.03; 7.25 Hz, 1H); 7.93 (dd, J=7.19, 7.95, 1H); 6.53 (br, 1H); 2.64 (d, J=7.06 Hz, 2H); 2.46 (s, 3H); 2.00˜1.91 (m, 1H); 0.92 (d, J=6.62 Hz, 6H). ¹³C NMR (DMSO-d₆, 75 MHz), δ 180.87; 166.64; 153.34; 149.61; 148.86; 145.90; 140.45; 134.37; 130.05; 129.91; 129.78; 128.99; 127.26; 124.05; 115.96; 107.06; 103.59; 32.15; 29.00; 23.37; 20.43.

### EXAMPLE 21

### 8-Bicyclo[2.2.1]hept-2-ylmethyl-5,6,7-trihydroxy-2-methyl-3-(4-phenoxy-phenyl)-chromen-4-one (Trans/Cis=3:1˜2:1)

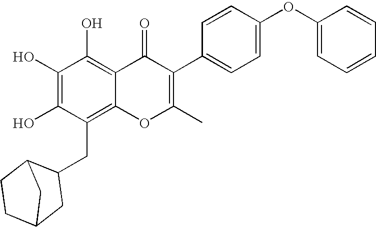

¹H NMR (CDCl₃, 300 MHz), δ 12.81 (s, 1H); 7.41˜7.36 (m, 2H); 7.27˜7.26 (m, 1H); 7.19˜7.04 (m, 6H); 6.15 (br, 1H); 5.49 (br, 1H); 2.92˜2.66 (m, 2H); 2.44˜2.20 (m, 5H); 2.06˜0.98 (m, 8H); 0.93˜0.85 (m, 2H); ¹³C NMR (CDCl₃, 75 MHz), δ 181.64; 164.14; 157.27; 156.67; 148.49; 131.86; 129.81; 126.81; 126.56; 123.65; 120.39; 119.45; 118.45; 106.91; 104.33; 40.50; 40.28; 39.94; 37.28; 36.11; 35.07; 31.59; 30.57; 24.90; 22.66; 22.58; 19.54.

### EXAMPLE 22

### 8-Biphenyl-4-yl-5,6,7-trihydroxy-2-methyl-3-(4-phenoxy-phenyl)-chromen-4-one

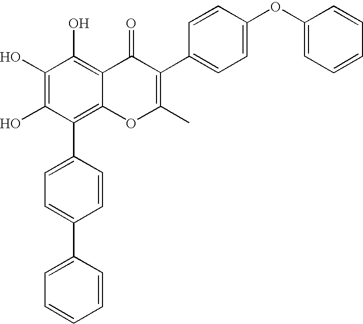

¹H NMR (CDCl₃, 300 MHz), δ 7.73˜7.67 (m, 4H); 7.61 (d, J=8.15 Hz, 2H); 7.48 (t, J=7.41 Hz, 2H); 7.41˜7.36 (m, 3H); 7.26 (d, J=8.67 Hz, 1H); 7.18˜7.07 (m, 6H); 2.23 (s, 3H); ¹³C NMR (CDCl₃, 75 MHz), δ 181.45; 164.23; 157.22; 156.67; 147.74; 145.46; 140.75; 140.05; 131.85; 131.33; 130.54; 129.80; 128.80; 127.33; 127.04; 126.80; 126.58; 123.63; 120.37; 119.40; 118.44; 107.48; 104.16; 19.49.

### EXAMPLE 23

### 5,6,7-Trihydroxy-2-methyl-3,8-bis-(4-phenoxy-phenyl)-chromen-4-one

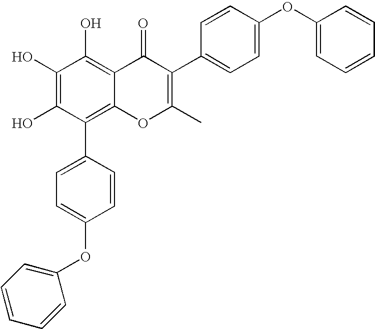

¹H NMR (DMSO-d₆, 300 MHz), δ 13.08 (s, 1H); 7.63˜7.08 (m, 18H); 6.28 (br 1H); 4.82 (br, 1H); 2.24 (s, 3H).

### EXAMPLE 24

### 5,6,7-Trihydroxy-8-(6-hydroxy-naphthalen-2-yl)-2-methyl-3-(4-phenoxy-phenyl)-chromen-4-one

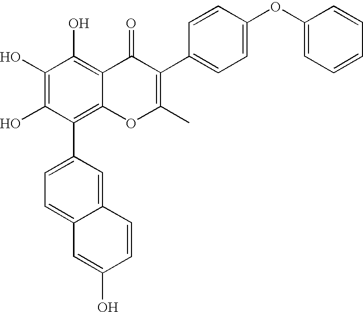

¹H NMR (DMSO-d₆, 300 MHz), δ 7.88 (s, 1H); 7.73 (d, J=8.89 Hz, 1H); 7.65 (d, J=8.90 Hz, 1H); 7.53 (d, J=8.53 Hz, 1H); 7.45˜7.40 (m, 3H); 7.31 (d, J=8.53 Hz, 2H); 7.22˜7.17 (m, 2H); 7.11˜7.02 (m, 4H); 2.09 (s, 3H).

### EXAMPLE 25

### 5,6,7-Trihydroxy-2-methyl-3-(4-phenoxy-phenyl)-8-phenyl-chromen-4-one

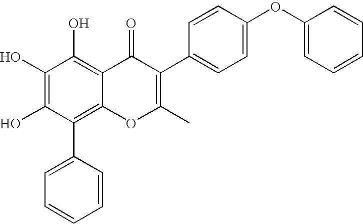

¹H NMR (CDCl₃, 300 MHz), δ 13.08 (s, 1H); 7.59˜7.35 (m, 7H); 7.28˜6.93 (m, 7H); 6.16 (s, 1H); 5.59 (s, 1H); 2.22 (s, 3H); ¹³C NMR (CDCl₃, 75 MHz), δ 182.02; 165.07; 157.78; 157.04; 148.12; 145.79; 132.21; 131.25; 131.09; 130.23; 128.83; 128.29; 127.76; 126.70; 124.10; 121.06; 119.88; 118.87; 108.11; 105.02; 19.89.

### EXAMPLE 26

### 5,6,7-Trihydroxy-2-methyl-3-(4-phenoxy-phenyl)-chromen-4-one

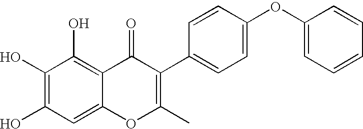

¹H NMR (DMSO-d₆, 300 MHz), δ 12.80 (s, 1H); 8.75 (br, 1H); 7.43 (t, J=7.63 Hz, 2H); 7.32˜7.29 (m, 2H); 7.17 (t; J=7.22 Hz, 1H); 7.09˜6.96 (m, 4H); 6.46 (s, 1H); 2.28 (s, 3H); ¹³C NMR (DMSO-d₆, 75 MHz), δ 181.24; 164.96; 157.21; 157.11; 154.25; 150.44; 148.02; 133.23; 131.00; 129.85; 127.92; 124.59; 120.23; 119.80; 118.85; 104.35; 94.16; 20.13.

### EXAMPLE 27

### 8-Bromo-5,6,7-trihydroxy-2-methyl-3-(4-phenoxy-phenyl)-chromen-4-one

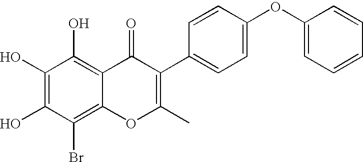

¹H NMR (CDCl₃, 300 MHz), δ 12.91 (s, 1H); 7.42˜7.09 (m, 9H); 6.55 (s, 1H); 5.73 (s, 1H); 2.34 (s, 3H).

### EXAMPLE 28

### 3-(6-Hydroxy-5-isobutyl-naphthalen-2-yl)-8-isobutyl-5,6,7-trimethoxy-2-methyl-chromen-4-one

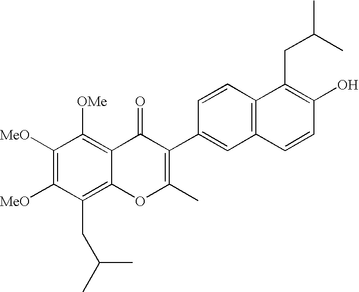

¹H NMR (CDCl₃, 300 MHz), δ 7.92 (d, J=8.76 Hz, 1H); 7.65 (d, J=1.58 z, 1H); 7.52 (d, J=8.70 Hz, 1H); 7.29 (dd, J=1.74, 8.78 Hz, 1H); 7.02 (d, J=8.76 Hz, 1H); 5.29 (s, 1H); 4.07 (s, 3H); 3.96 (s, 3H); 3.93 (s, 3H); 2.90 (d, J=7.24 Hz, 2H); 2.74 (d, J=7.22 Hz, 2H); 2.32 (s, 3H); 2.13˜1.96 (m, 2H); 1.03 d, J=6.67 Hz, 6H); 1.00 (d, J=6.68 Hz, 6H).

### EXAMPLE 29

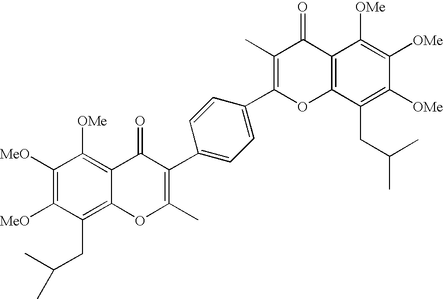

¹H NMR (DMSO-d₆, 300 MHz), δ 7.37 (s, 4H); 3.96 (s, 6H); 3.90 (s, 6H); 3.87 (s, 6H); 2.72 (d, J=7.16 Hz, 4H); 2.33 (s, 6H); 2.06˜1.95 (m, 2H); 0.99 (d, J=6.64 Hz, 12H).

### EXAMPLE 30

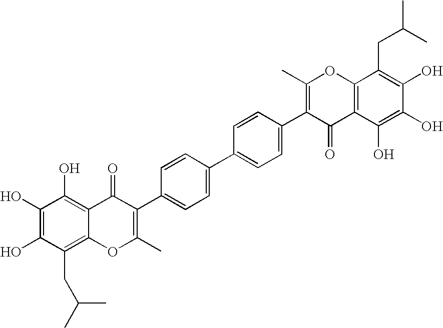

¹H NMR (DMSO-d₆, 300 MHz), δ 12.85 (s, 2H); 9.86 (s, 2H); 9.13 (s, 2H); 7.80 (d, J=8.28 Hz, 4H); 7.46 (d, J=8.21 Hz, 4H); 2.62 (d, J=6.98 Hz, 4H); 2.35 (s, 6H); 1.99˜1.90 (m, 2H); 0.91 (d, J=6.63 Hz, 12H); ¹³C NMR (DMSO-d₆, 75 MHz), δ 181.73; 164.96; 152.93; 148.92; 146.09; 140.01; 132.59; 132.36; 129.26; 127.39; 120.35; 106.60; 104.01; 32.24; 31.73; 23.49; 20.41.

### EXAMPLE 31

### N-Benzyl-3-(5,6,7-trihydroxy-8-isobutyl-2-methyl-4-oxo-4H-chromen-3-yl)-benzamide

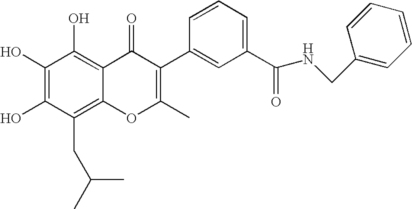

¹H NMR (DMSO-d₆, 300 MHz), δ 12.74 (s, 1H); 9.82 (s, 1H); 9.11 (s, 1H); 9.06 (t, J=6.00 Hz, 1H); 7.94˜7.91 (m, 1H); 7.86 (s, 1H); 7.57˜7.47 (m, 2H); 7.33˜7.22 (m, 4H); 4.49 (d, J=5.88 Hz, 2H); 2.60 (d, J=7.15 Hz, 2H); 2.28 (s, 3H); 1.98˜1.89 (m, 1H); 0.90 (d, J=6.64 Hz, 6H); ¹³C NMR (DMSO-d₆, 75 MHz), δ 181.51; 166.76; 164.85; 152.88; 148.87; 145.97; 140.56; 135.12; 134.40; 133.43; 130.42; 129.22; 129.18; 128.22; 127.66; 127.57; 120.27; 106.58; 103.89; 43.64; 32.13; 31.63; 23.39; 20.23.

### EXAMPLE 32

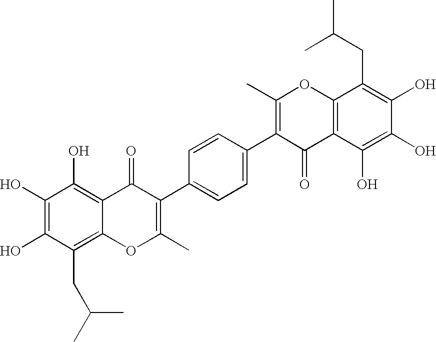

¹H NMR (DMSO-d₆, 300 MHz), δ 12.85 (s, 2H); 9.85 (s, 2H); 9.13 (s, 2H); 7.40 (s, 4H); 2.61 (d, J=7.03 Hz, 4H); 2.33 (s, 6H); 1.99˜1.88 (s, 2H); 0.91 (d, J=6.63 Hz, 12H); ¹³C NMR (DMSO-d₆, 75 MHz), δ 181.69; 164.97; 152.90; 148.89; 146.07; 132.56; 131.39; 129.23; 120.47; 106.57; 104.00; 32.21; 29.03; 23.47; 20.36.

### EXAMPLE 33

### 4-(8-Isobutyl-5,6,7-trimethoxy-2-methyl-4-oxo-4H-chromen-3-yl)-benzoic acid methyl ester

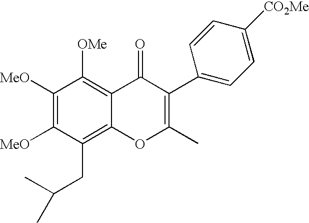

¹H NMR (CDCl₃, 300 MHz), δ 8.11 (dd, J=1.81, 6.45 Hz, 2H); 7.40 (d, J=6.43 Hz, 2H); 4.02 (s, 3H); 3.95 (s, 3H); 3.90 (s, 3H); 3.88 (s, 3H); 2.72 (d, J=7.20 Hz, 2H); 2.28 (s, 3H); 2.00˜1.91 (m, 1H); 0.89 (d, J=6.98 Hz, 6H).

### EXAMPLE 34

### 4-(8-Isobutyl-5,6,7-trimethoxy-2-methyl-4-oxo-4H-chromen-3-yl)-N-(3-isopropyl-phenyl)-benzamide

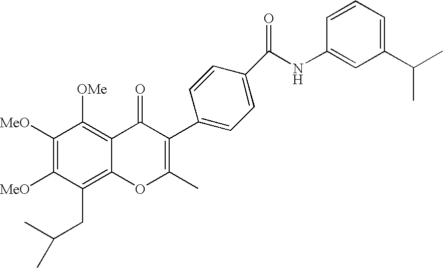

¹H NMR (CDCl₃, 300 MHz), δ 8.29 (s, 1H); 7.89 (d, J=8.06 Hz, 2H); 7.58˜7.54 (m, 2H); 7.36˜7.26 (m, 3H); 7.02 (d, J=7.27 Hz, 1H); 4.03 (s, 3H); 3.94 (s, 3H): 3.88 (s, 3H); 2.96˜2.84 (m, 1H); 2.73 (d, J=7.10 Hz, 2H); 2.27 (s, 3H): 2.00˜1.94 (m, 1H); 1.23 (d, J=7.00 Hz, 6H); 0.91 (d, J=6.39 Hz, 6H).

### EXAMPLE 35

### 4-(5,6,7-Trihydroxy-8-isobutyl-2-methyl-4-oxo-4H-chromen-3-yl)-benzoic acid

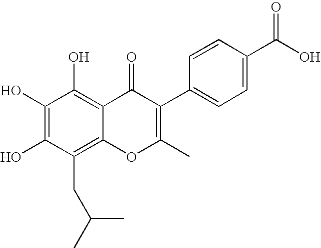

¹H NMR (DMSO-d₆, 300 MHz), δ 13.03 (br, 1H); 12.66 (s, 1H); 10.19 (s, 1H); 9.15 (Br, 1H); 7.99 (d, J=8.19 Hz, 2H); 7.48 (d, J=8.18 Hz, 2H); 2.60 (d, J=7.20 Hz, 2H); 2.22 (s, 3H); 2.00˜1.91 (m, 1H); 0.90 (d, J=6.66 Hz, 6H); ¹³C NMR (DMSO-d₆, 75 MHz), δ 181.20; 168.02; 164.91; 152.86; 148.74; 145.91; 137.94; 131.87; 130.77; 129.86; 129.19; 106.54; 103.78; 32.06; 28.90; 23.32; 20.16.

### EXAMPLE 36

### 3-(8-Isobutyl-5,6,7-trimethoxy-2-methyl-4-oxo-4H-chromen-3-yl)-N-(2-isopropyl-phenyl)-benzamide

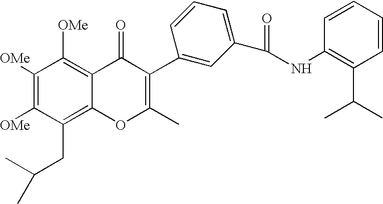

¹H NMR (CDCl₃, 300 MHz), δ 7.90˜7.75 (m, 3H); 7.62˜7.50 (m, 2H); 7.38˜7.35 (m, 1H); 7.26˜7.22 (m, 4H); 4.03 (s, 3H); 3.95 (s, 3H); 3.89 (s, 3H); 3.19˜3.10 (m, 1H); 2.73 (d, J=7.10 Hz, 2H); 2.38 (s, 3H); 2.01˜1.94 (m, 1H); 1.29 (d, J=6.84 Hz, 6H); 0.99 (d, J=6.64 Hz, 6H).

### EXAMPLE 37

### 4-(6,7-Bis-ethoxymethoxy-5-hydroxy-8-isobutyl-2-methyl-4-oxo-4H-chromen-3-yl)-N-phenyl-benzamide

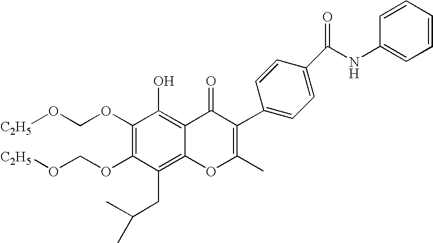

¹H NMR (CDCl₃, 300 MHz), δ 12.80 (s, 1H); 7.95 (d, J=8.34 Hz, 2H); 7.94 (s, 1H); 7.69 (d, J=8.54 Hz, 2H); 7.44˜7.35 (m, 4H); 7.19 (t, J=7.49 Hz, 1H); 5.36 (s, 2H); 5.25 (s, 2H); 3.94˜3.91 (m, 4H); 2.73 (d, J=7.18 Hz, 2H); 2.34 (s, 3H); 2.07˜1.95 (m, 1H); 1.29 (t, J=7.08 Hz, 3H); 1.24 (t, J=7.06 Hz H); 0.97 (d, J=6.63 Hz, 6H).

### EXAMPLE 38

### 4-(6,7-Bis-ethoxymethoxy-5-hydroxy-8-isobutyl-2-methyl-4-oxo-4H-chromen-3-yl)-benzoic acid

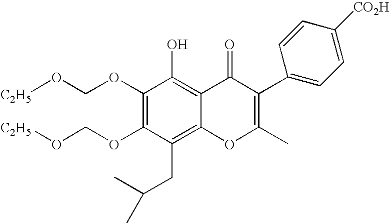

¹H NMR (Acetone-d₆, 300 MHz), δ 13.07 (s, 1H); 8.14 (d, J=8.49 Hz, 2H); 7.56 (d, J=8.41 Hz, 2H); 5.37 (s, 2H); 5.22 (s, 2H); 3.90˜3.82 (m, 4H); 2.78 (d, J=7.21 Hz, 2H); 2.42 (s, 3H); 2.01˜1.95 (m, 1H); 1.28˜1.12 (m, 6H); 0.98 (d, J=6.66 Hz, 6H).

### EXAMPLE 39

### (S)4-Methyl-2-[4-(5,6,7-trihydroxy-8-isobutyl-2-methyl-4-oxo-4H-chromen-3-yl)-benzoylamino]-pentanoic acid methyl ester

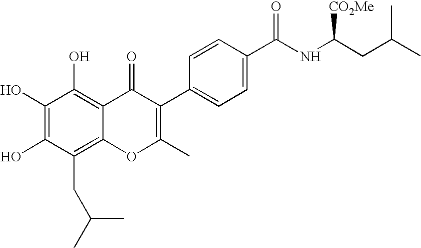

¹H NMR (DMSO-d₆, 300 MHz), δ 12.75 (s, 1H); 9.88 (s, 1H); 9.14 (s, 1H); 8.80 (d, J=7.68 Hz, 1H); 7.95 (d, J=8.28 Hz, 2H); 7.46 (d, J=8.28 Hz, 2H); 4.55˜4.52 (m, 1H); 3.66 (s, 3H); 2.60 (d, J=7.12 Hz, 2H); 2.28 (s, 3H); 1.99˜1.55 (m, 4H); 0.90 (d, J=6.64 Hz, 6H); 0.84 (d, J=6.76 Hz, 6H); ¹³C NMR (DMSO-d₆, 75 MHz), δ 181.10; 173.79; 167.00; 164.65; 152.63; 148.55; 145.72; 136.33; 133.51; 131.34; 128.96; 127.93; 119.80; 106.31; 103.59; 52.58; 51.60; 31.86; 28.69; 25.15; 23.55; 23.11; 21.80; 19.93.

### EXAMPLE 40

### N-(1-Benzyl-piperidin-4-yl)-4-(5,6,7-trihydroxy-8-isobutyl-2-methyl-4-oxo-4H-chromen-3-yl)-benzamide: hydrochloride salt

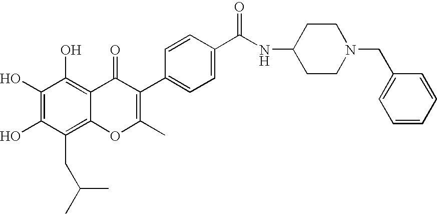

¹H NMR (DMSO-d₆, 300 MHz), δ 12.72 (s, 1H); 10.85 (s, 1H); 9.88 (s, 1H); 9.30 (s, 1H); 8.66 (s, 1H); 7.93 (br, 2H); 7.64 (br, 2H); 7.46 (m, 5H); 4.26 (s, 2H); 3.90 (m, 1H); 3.45 (m, 2H); 3.07 (m, 2H); 2.58 (m, 2H); 2.26 (s, 3H); 2.08˜1.90 (m, 5H); 0.90 (d, J=6.65 Hz, 6H); ¹³C NMR (DMSO-d₆, 75 MHz), δ 181.30; 166.58; 164.81; 152.79; 148.72; 145.96; 136.25; 134.25; 132.37; 131.44; 130.77; 130.32; 129.64; 129.17; 128.03; 120.00; 106.51; 103.80; 59.79; 51.50; 45.77; 32.06; 29.26; 28.88; 23.32; 20.15.

### EXAMPLE 41

### (S) 3-(1H-Indol-2-yl)-2-[4-(5,6,7-trihydroxy-8-isobutyl-2-methyl-4-oxo-4H-chromen-3-yl)-benzoylamino]-propionic acid methyl ester

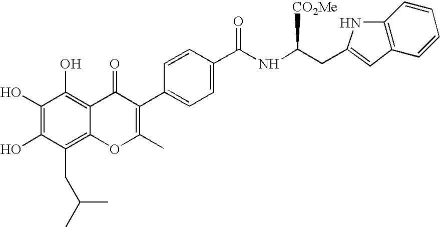

¹H NMR (DMSO-d₆, 300 MHz), δ 12.74 (s, 1H); 10.93 (s, 1H); 9.87 (s, 1H); 9.14 (br, 1H); 8.89 (d, J=7.61 Hz, 1H); 7.89 (d, J=8.28 Hz, 2H); 7.54 (d, J=7.74 Hz, 1H); 7.43 (d, J=8.27 Hz, 2H); 7.34 (d, J=7.87 Hz, 1H); 7.24 (d, J=2.00 Hz, 1H); 7.10˜6.94 (m, 2H); 4.76˜4.68 (m, 1H); 3.66 (s, 3H); 3.34˜3.21 (m, 2H); 2.60 (d, J=7.20 Hz, 2H); 2.22 (s, 3H); 1.98˜1.91 (m, 1H); 0.90 (d, J=6.63 Hz, 6H); ¹³C NMR (DMSO-d₆, 75 MHz), δ 181.09; 173.23; 166.88; 164.66; 152.64; 148.55; 145.73; 136.78; 136.32; 133.49; 131.33; 128.96; 127.86; 127.72; 124.34; 121.67; 119.78; 119.12; 118.67; 112.16; 110.64; 106.32; 103.60; 54.50; 52.63; 31.87; 28.70; 27.29; 22.74; 19.95.

### EXAMPLE 42

### (S) Phenyl-[4-(5,6,7-trihydroxy-8-isobutyl-2-methyl-4-oxo-4H-chromen-3-yl)-benzoylamino]-acetic acid methyl ester

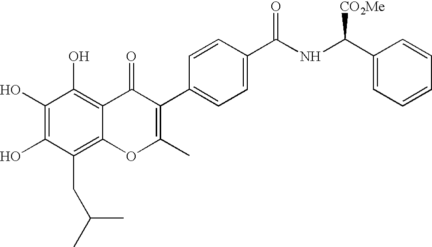

¹H NMR (DMSO-d₆, 300 MHz), δ 12.75 (s, 1H); 9.88 (s, 1H); 9.28 (d, J=7.14 Hz, 1H); 9.14 (s, 1H); 7.99 (d, J=8.28 Hz, 2H); 7.52˜7.35 (m, 7H); 5.71 (d, J=7.08 Hz, 1H); 3.68 (s, 3H); 2.60 (d, J=7.07 Hz, 2H); 2.28 (s, 3H); 1.98˜1.89 (m, 1H); 0.90 (d, J=6.63 Hz, 6H); ¹³C NMR (DMSO-d₆, 75 MHz), δ 181.08; 171.74; 166.96; 164.64; 152.61; 148.53; 145.72; 136.85; 136.42; 133.30; 129.21; 128.94; 128.87; 128.18; 119.78; 106.30; 103.59; 57.57; 52.96; 31.84; 28.68; 23.10; 22.73; 19.92.

### EXAMPLE 43

### N-[2-(1H-Indol-3-yl)-ethyl]-4-(5,6,7-trihydroxy-8-isobutyl-2-methyl-4-oxo-4H-chromen-3-yl)-benzamide

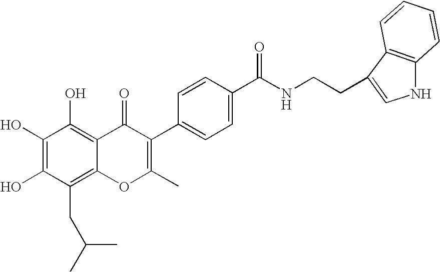

¹H NMR (DMSO-d₆, 300 MHz), δ 12.76 (s, 1H); 10.83 (s, 1H); 9.87 (s, 1H); 9.13 (br, 1H); 8.69 (t, J=5.50 Hz, 1H); 7.91 (d, J=8.25 Hz, 2H); 7.60 (d, J=7.50 Hz, 1H); 7.43 (d, J=8.25 Hz, 2H); 7.35 (d, I=8.01 Hz, 1H); 7.20 (s, 1H); 7.13˜6.97 (m, 2H); 3.59˜3.50 (m, 2H); 2.98 (t, J=7.50 Hz, 2H); 2.60 (d, J=7.20 Hz, 2H); 2.29 (s, 3H); 1.96˜1.91 (m, 1H); 0.90 (d, J=6.60 Hz, 6H).

### EXAMPLE 44

### N-Benzhydryl-4-(5,6,7-trihydroxy-8-isobutyl-2-methyl-4-oxo-4H-chromen-3-yl)-benzamide

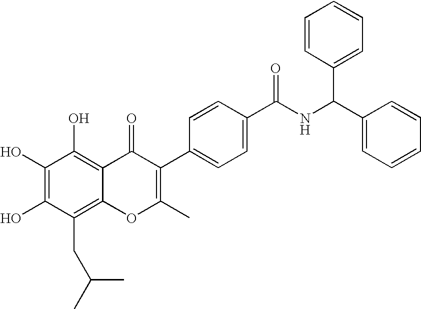

¹H NMR (DMSO-d₆, 300 MHz), δ 12.75 (s, 1H); 9.87 (s, 1H); 9.36 (d, J=8.80 Hz, 1H); 9.12 (s, 1H); 8.00 (d, J=8.38 Hz, 2H); 7.46˜7.26 (m, 12H); 6.45 (d, J=8.64 Hz, 1H); 2.60 (d, J=7.48 Hz, 2H); 2.28 (s, 3H); 1.98˜1.91 (m, 1H); 0.84 (d, J=6.46 Hz, 6H); ¹³C NMR (DMSO-d₆, 75 MHz), δ 181.33; 166.60; 164.84; 152.82; 148.75; 145.93; 143.18; 136.34; 131.46; 129.22; 128.50; 128.29; 127.87; 120.04; 106.50; 103.80; 57.22; 35.05; 28.89; 23.32; 20.14.

### EXAMPLE 45

### 3-(5,6,7-Trihydroxy-8-isobutyl-2-methyl-4-oxo-4H-chromen-3-yl)-benzoic acid

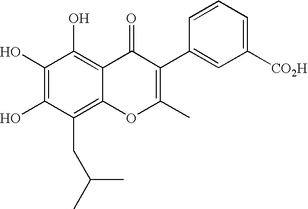

¹H NMR (DMSO-d₆, 300 MHz), δ 12.69 (br, 1H); 9.75 (br, 1H); 7.95 (s, 1H); 7.89 (m, 1H); 7.57˜7.49, 2H); 2.60 (d, J=6.70 Hz, 2H); 2.28 (s, 3H); 1.96˜1.91 (m, 1H); 0.90 (d, J=6.35 Hz, 6H).

### EXAMPLE 46

### N-Benzyl-4-(5,6,7-trihydroxy-8-isobutyl-2-methyl-4-oxo-4H-chromen-3-yl)-benzamide

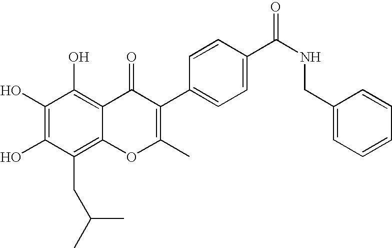

¹H NMR (DMSO-d₆, 300 MHz), δ 12.75 (s, 1H); 9.87 (s, 1H); 9.14 (s, 1H); 9.12 (s, 1H); 7.96 (d, J=8.25 Hz, 2H); 7.42 (d, J=8.24 Hz, 2H); 7.41˜7.24 M, 5H); 4.51 (d, J=5.84 Hz, 2H); 2.60 (d, J=7.11 Hz, 2H); 2.28 (s, 3H); 1.97˜1.89 (m, 1H); 0.90 (d, J=6.61 Hz, 6H); ¹³C NMR (DMSO-d₆, 75 MHz), δ 181.46; 167.00; 165.01; 152.95; 148.89; 146.05; 140.67; 136.41; 134.46; 131.73; 129.30; 128.17; 128.02; 127.75; 120.14; 106.65; 103.94; 43.61; 32.18; 31.51; 29.03; 23.45; 20.28.

### EXAMPLE 47

### (S) 3-Phenyl-2-[4-(5,6,7-trihydroxy-8-isobutyl-2-methyl-4-oxo-4H-chromen-3-yl)-benzoylamino]-propionic acid methyl ester

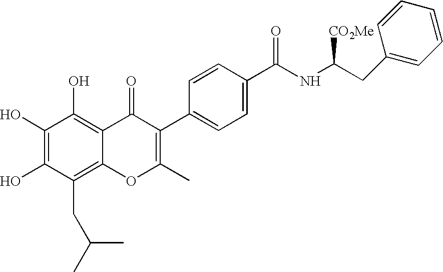

¹H NMR (DMSO-d₆, 300 MHz), δ 12.74 (s, 1H); 9.88 (s, 1H); 9.13 (br, 1H); 8.93 (d, J=7.85 Hz, 1H); 7.86 (d, J=8.28 Hz, 2H); 7.43 (d, J=8.26 Hz, 2H); 7.39˜7.18 (m, 5H); 4.70˜4.68 (m, 1H); 3.66 (s, 3H); 3.19˜3.12 (m, 2H); 2.60 (d, J=7.14 Hz, 2H); 2.27 (s, 3H); 1.98˜1.89 (m, 1H); 0.90 (d, J=6.63 Hz, 6H); ¹³C NMR (DMSO-d₆, 75 MHz), δ 181.07; 172.85; 166.84; 164.63; 152.62; 148.52; 145.71; 138.40; 136.34; 133.41; 131.34; 129.73; 128.92; 127.79; 127.16; 119.75; 106.30; 103.58; 54.96; 52.65; 36.85; 31.85; 22.72; 19.93.

### EXAMPLE 48

### 5,6,7-Trihydroxy-2-methyl-8-naphthalen-2-yl-3-(4-phenoxy-phenyl)-chromen-4-one

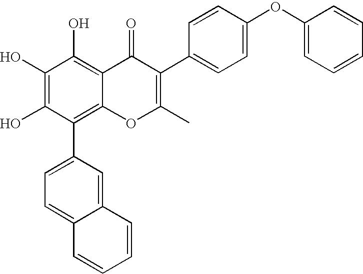

¹H NMR (CDCl₃, 300 MHz), δ 13.10 (s, 1H); 8.00˜7.90 (m, 3H); 7.63˜7.50 (m, 2H); 7.40˜7.35 (m, 2H); 7.28˜7.18 (m, 4H); 7.16˜6.93 (m, 5H); 6.22 (br, 1H); 5.57 (br, 1H); 2.25 (s, 3H); ¹³C NMR (CDCl₃, 75 MHz), δ 181.61; 164.70; 157.36; 156.60; 147.85; 145.50; 133.37; 132.81; 131.79; 130.01; 129.82; 128.63; 128.17; 128.09; 127.93; 127.74; 127.40; 126.37; 126.21; 123.68; 120.69; 119.46; 118.46; 107.51; 104.66; 19.50.

### EXAMPLE 49

### 8-Cyclobutylmethyl-5,6,7-trihydroxy-2-methyl-3-(4-phenoxy-phenyl)-chromen-4-one

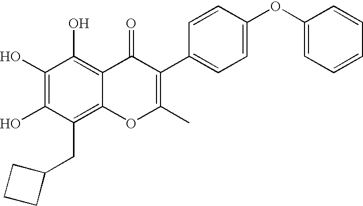

¹H NMR (CDCl₃, 300 MHz), δ 12.79 (s, 1H); 7.41˜7.03 (m, 7H); 6.96 (dd, J1=2.2 Hz, J2=7.69 Hz, 1H); 6.83 (d, J=8.71 Hz, 1H); 6.13 (s, 1H); 5.39 (s, 1H); 2.92 (d, J=7.37 Hz, 2H); 2.69˜2.64 (m, 1H); 2.38 (s, 3H); 2.04˜1.77 (m, 6H); ¹³C NMR (CDCl₃, 75 MHz), δ 181.44; 157.26; 156.66; 143.72; 131.84; 129.81; 129.61; 126.83; 123.64; 122.45; 121.00; 119.44; 118.46; 117.58; 116.28; 105.78; 104.33; 135.97; 28.90; 28.20; 19.60; 18.29.

### EXAMPLE 50

### 2-Cyclopropyl-5,6,7-trihydroxy-8-isobutyl-3-(4-phenoxy-phenyl)-chromen-4-one

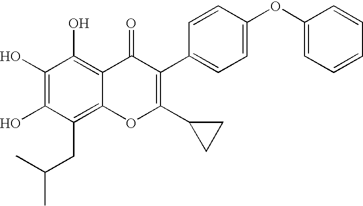

¹H NMR (CDCl₃, 300 MHz), δ 12.93 (s, 1H); 7.41˜7.06 (m, 7H); 6.97 (dd, J=1.76, 8.61 Hz, 1H); 6.83 (d, J=8.70 Hz, 1H); 6.11 (s, 1H); 5.43 (s, 1H); 2.60 (d, J=7.26 Hz, 2H); 2.03˜1.89 (m, 2H); 1.35˜1.29 (m, 2H); 1.08˜1.02 (m, 2H); 0.98 (d, J=6.64 Hz, 6H); ¹³C NMR (CDCl₃, 75 MHz), δ 180.96; 167.13; 157.24; 156.70; 148.64; 147.97; 132.36; 129.82; 129.62; 126.80; 126.39; 123.64; 122.47; 121.01; 119.47; 119.35; 118.46; 117.60; 116.31; 106.26; 31.61; 28.92; 22.62; 13.36; 9.47.

### EXAMPLE 51

### 5,6,7-Trihydroxy-8-isobutyl-2-methyl-3-[4-(naphthalen-2-yloxy)-phenyl]-chromen-4-one

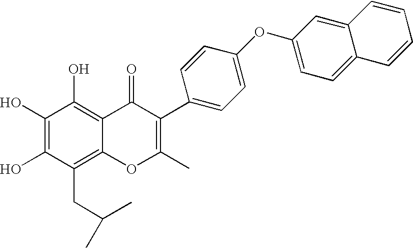

¹H NMR (CDCl₃, 300 MHz), δ 12.82 (s, 1H); 7.87 (dd, J=8.74, 8.64 Hz, 1H); 7.77 (d, J=7.66 Hz, 1H); 7.53˜7.42 (m, 2H); 7.38˜7.28 (m, 5H); 7.21˜7.14 (m, 2H); 6.15 (s, 1H); 5.44 (s, 1H); 2.71 (d, J=7.25 Hz, 2H); 2.36 (s, 3H); 2.09˜1.99 (m, 1H); 0.99 (d, J=6.63 Hz, 6H); ¹³C NMR (CDCl₃, 75 MHz), δ 181.65; 164.20; 157.22; 154.41; 148.86; 143.77; 134.31; 131.92; 130.37; 129.96; 127.75; 127.20; 126.76; 126.56; 124.87; 120.27; 118.64; 114.91; 106.34; 31.37; 28.64; 22.55; 19.58.

### EXAMPLE 52

### 3-Biphenyl-4-yl-5,6,7-trihydroxy-8-isobutyl-2-methyl-chromen-4-one

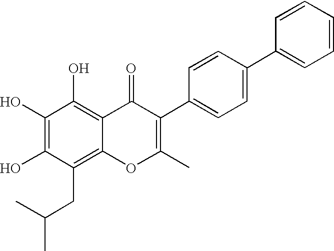

¹H NMR (CDCl₃, 300 MHz), δ 12.82 (s, 1H); 7.71˜7.64 (m, 4H); 7.51˜7.39 (m, 5H); 6.13 (s, 1H); 5.35 (s, 1H); 2.73 (d, J=7.18 Hz, 2H); 2.40 (s, 3H); 2.06˜2.02 (m, 1H); 0.99 (d, J=6.64 Hz, 6H); ¹³C NMR (CDCl₃, 75 MHz), δ 181.60; 164.12; 148.81; 143.82; 140.89; 140.80; 130.98; 130.83; 128.80; 127.41; 127.32; 127.18; 126.84; 123.58; 120.64; 106.35; 31.38; 28.65; 22.56; 19.59.

### EXAMPLE 53

### 3-(4-Benzenesulfonyl-phenyl)-5,6,7-trihydroxy-8-isobutyl-2-methyl-chromen-4-one

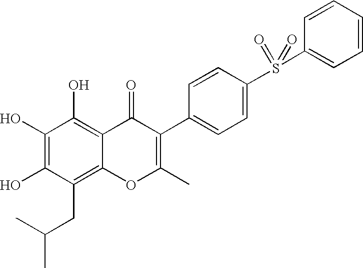

¹H NMR (CDCl₃, 300 MHz), δ 12.52 (s, 1H); 8.06˜8.00 (m, 4H); 7.61˜7.45 (m, 5H); 6.31 (s, 1H); 5.84 (s, 1H); 2.69 (d, J=6.91 Hz, 2H); 2.32 (s, 3H); 2.05˜1.95 (m, 1H); 0.97 (d, J=6.42 Hz, 6H).

### EXAMPLE 54

### N-Adamantan-1-yl-4-(5,6,7-trihydroxy-8-isobutyl-2-methyl-4-oxo-4H-chromen-3-yl)-benzamide

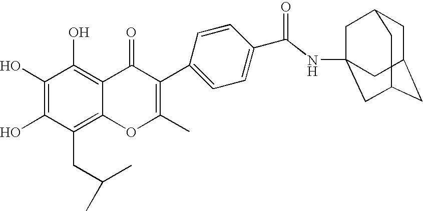

¹H NMR (CDCl₃, 300 MHz), δ 12.71 (s, 1H); 7.81 (d, J=8.25 Hz, 2H); 7.38 (d, J=8.25 Hz, 2H); 6.20 (s, 1H); 5.83 (s, 1H); 5.55 (s, 1H); 2.70 (d, J=7.23 Hz, 2H); 2.28 (s, 3H); 2.19˜1.98 (m, 4H); 1.76˜1.66 (m, 12H); 0.98 (d, J=6.64 Hz, 6H); ¹³C NMR (CDCl₃, 75 MHz), δ 181.03; 166.57; 163.89; 148.75; 148.74; 135.87; 135.02; 130.67; 126.90; 120.16; 106.08; 103.82; 52.34; 41.66; 36.35; 31.36; 29.47; 28.63; 22.53; 19.47.

### EXAMPLE 55

### 3-(4-Chloro-phenyl)-5,6,7-trihydroxy-8-isobutyl-2-methyl-chromen-4-one

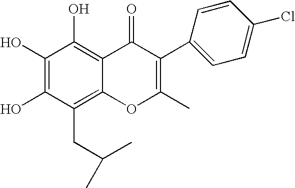

¹H NMR (CDCl₃, 300 MHz), δ 12.77 (s, 1H); 7.45 (d, J=8.40 Hz, 2H); 7.26 (d, J=8.40 Hz, 2H); 6.35 (s, 1H); 5.98 (s, 1H); 2.69 (d, J=7.11 Hz, 2H); 2.33 (s, 3H); 2.06˜1.94 (m, 1H); 0.97 (d, J=6.63 Hz, 6H); ¹³C NMR (CDCl₃, 75 MHz), δ 181.29; 164.12; 149.35; 148.83; 143.73; 134.05; 131.87; 130.50; 128.77; 127.06; 119.88; 106.45; 104.16; 31.36; 28.60; 22.53; 19.47.

### EXAMPLE 56

### 5,6,7-Trihydroxy-3-(4-hydroxy-phenyl)-8-isobutyl-2-methyl-chromen-4-one

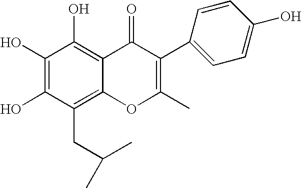

¹H NMR (DMSO-d₆, 300 MHz), δ 12.92 (s, 1H); 9.77 (br, 1H); 9.53 (br, 1H); 9.09 (br, 1H); 7.10 (d, J=8.52 Hz, 2H); 6.81 (d, J=8.55 Hz, 2H); 2.58 (d, J=6.99 Hz, 2H); 2.26 (s, 3H); 1.96˜1.88 (m, 1H); 0.90 (d, J=6.63 Hz, 6H); ³C NMR (DMSO-d₆, 75 MHz), δ 181.67; 164.32; 157.47; 152.40; 148.53; 145.72; 132.41; 128.74; 123.08; 120.27; 115.56; 106.06; 103.67; 31.86; 28.68; 23.12; 19.93.

### EXAMPLE 57

### 5,6,7-Trihydroxy-8-isobutyl-2-methyl-3-(4-phenoxy-phenyl)-chromen-4-one

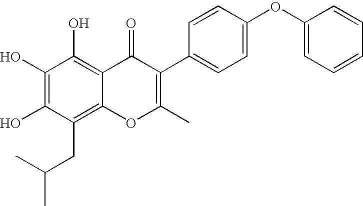

¹H NMR (DMSO-d₆, 300 MHz), δ 12.83 (s, 1H); 9.83 (br, 1H); 9.11 (br, 1H); 7.46˜7.33 (m, 4H); 7.21˜7.04 (m, 5H); 2.59 (d, J=7.18 Hz, 2H); 2.30 (s, 3H); 1.95˜1.91 (m, 1H); 0.90 (d, J=6.61 Hz, 6H); ¹³C NMR (DMSO-d₆, 75 MHz), δ 181.39; 164.59; 157.06; 156.87; 152.52; 148.54; 145.70; 133.04; 130.79; 128.86; 127.82; 124.35; 119.74; 119.58; 118.65; 106.19; 103.62; 31.85; 28.68; 23.12; 19.98.

### EXAMPLE 58

### N-Naphthalen-2-yl-4-(5,6,7-trihydroxy-8-isobutyl-2-methyl-4-oxo-4H-chromen-3-yl)-benzamide

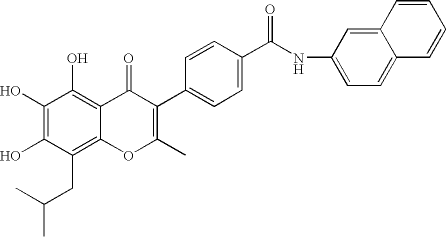

¹H NMR (DMSO-d₆, 300 MHz), δ 12.77 (s, 1H); 10.54 (s, 1H); 9.89 (s, 1H); 8.51 (br, 1H); 8.07 (d, J=8.27 Hz, 2H); 7.94˜7.84 (m, 4H); 7.54 (d, J=8.30 Hz, 2H); 7.89˜7.84 (m, 2H); 2.62 (d, J=7.08 Hz, 2H); 2.32 (s, 3H); 1.98˜1.90 (m, 1H); 0.91 (d, J=6.63 Hz, 6H); ¹³C NMR (DMSO-d₆, 75 MHz), δ 181.12; 166.26; 164.69; 152.65; 148.56; 145.74; 137.48; 136.49; 134.76; 134.00; 131.49; 130.65; 128.98; 128.85; 128.14; 128.10; 127.07; 125.46; 121.58; 119.78; 117.16; 106.33; 103.62; 31.87; 28.70; 23.13; 19.99.

### EXAMPLE 59

### (S) 2-[4-(5,6,7-Trihydroxy-8-isobutyl-2-methyl-4-oxo-4H-chromen-3-yl)-benzoylamino]-succinic acid dimethyl ester

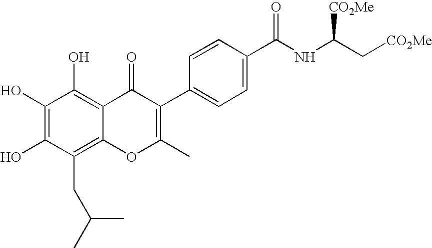

¹H NMR (DMSO-d₆, 300 MHz), δ 12.74 (s, 1H); 9.87 (s, 1H); 9.14 (br, 1H); 9.01 (d, J=7.68 Hz, 1H); 7.91 (d, J=8.31 Hz, 2H); 7.46 (d, J=8.35 Hz, 2H); 4.91˜4.84 (m, 1H); 3.68 (s, 3H); 3.64 (s, 3H); 3.03˜2.82 (m, 2H); 2.60 (d, J=7.07 Hz, 2H); 2.28 (s, 3H); 1.98˜1.86 (m, 1H); 0.95 (d, J=6.60 Hz, 6H).

### EXAMPLE 60

### 5,6,7-Trihydroxy-8-isobutyl-3-{4-[4-(3-methoxy-phenyl)-piperazine-1-carbonyl]-phenyl}-2-methyl-chromen-4-one (HCl)

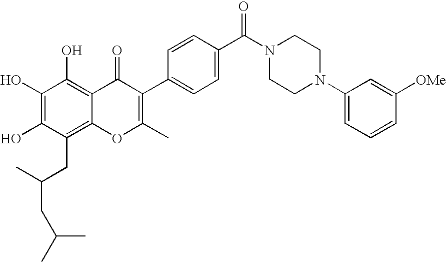

¹H NMR (DMSO-d₆, 300 MHz), δ 12.76 (br, 1H); 9.90 (br, 1H); 7.56 (d, J=8.20 Hz, 2H); 7.43 (d, J=8.18 Hz, 2H); 7.21 (dd, J=8.13, 8.16 Hz, 1H); 6.75 (s, 1H); 6.72 (d, J=7.76 Hz, 1H); 6.54 (d, J=7.77 Hz, 1H); 3.74 (s, 3H); 3.90˜3.57 (m, 8H); 2.60 (d, J=7.20 Hz, 2H); 2.31 (s, 3H); 2.00˜1.90 (m, 1H); 0.90 (d, J=6.63 Hz, 6H).

### EXAMPLE 61

### N-(2,2-Diphenyl-ethyl)-4-(5,6,7-trihydroxy-8-isobutyl-2-methyl-4-oxo-4H-chromen-3-yl)-benzamide

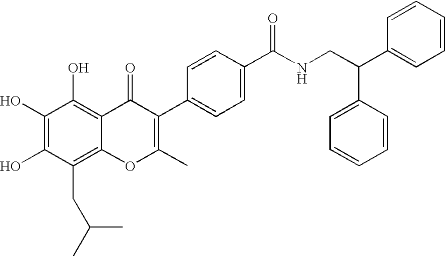

¹H NMR (DMSO-d₆, 300 MHz), δ 12.74 (s, 1H); 9.87 (s, 1H); 9.13 (br, 1H); 8.50 (t, J=5.15 Hz, 1H); 7.76 (d, J=8.24 Hz, 2H); 7.39˜7.17 (m, 12H); 4.56 (dd, J=8.03, 7.77 Hz, 1H); 3.93 (dd, J=5.61, 7.34 Hz, 2H); 2.59 (d, J=7.02 Hz, 2H); 2.25 (s, 3H); 1.97˜1.87 (m, 1H); 0.89 (d, J=6.62 Hz, 6H); ¹³C NMR (DMSO-d₆, 75 MHz), δ 181.08; 166.72; 164.58; 152.60; 148.51; 145.50; 143.55; 135.84; 134.35; 131.22; 129.07; 128.93; 128.60; 127.48; 126.99; 119.77; 106.28; 103.57; 50.50; 44.46; 31.84; 28.67; 23.09; 19.92.

### EXAMPLE 62

### 3-[4-(4-Benzyl-[1,4]diazepane-1-carbonyl)-phenyl]-5,6,7-trihydroxy-8-isobutyl-2-methyl-chromen-4-one Hydrochloride

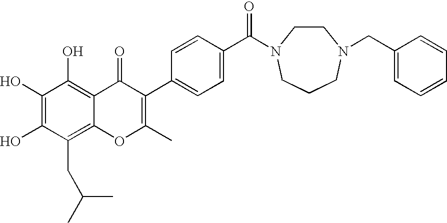

¹H NMR (DMSO-d₆, 300 MHz), δ 12.75 (s, 1H); 10.84 (br, 1H); 9.89 (s, 1H); 9.16 (br, 1H); 7.64˜7.41 (m, 9H); 5.76 (s, 2H); 4.41˜3.15 (m, 8H); 2.55 (d, J=6.83 Hz, 2H); 2.30 (s, 3H); 2.20˜2.00 (m, 2H); 1.99˜1.87 (m, 1H); 0.90 (d, J=6.67 Hz, 6H).

### EXAMPLE 63

### N-[1-Benzyl-2-(4-methyl-piperazin-1-yl)-2-oxo-ethyl]-4-(5,6,7-trihydroxy-8-isobutyl-2-methyl-4-oxo-4H-chromen-3-yl)-benzamide

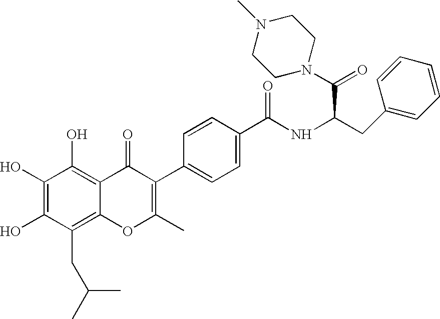

¹H NMR (DMSO-d₆, 300 MHz), δ 10.12 (br, 1H); 9.50 (br, 1H); 9.13 (s, 1H); 8.93˜18 8.87 (m, 1H); 7.86 (d, J=8.04 Hz, 2H); 7.43 (d, J=8.10 Hz, 2H); 7.32˜7.21 (m, 5H); 4.68˜4.58 (m, 1H); 4.02 (t, J=6.20 Hz, 1H); 3.90˜2.55(m, 10H); 2.80 (s, 3H); 2.60 (d, J=6.90 Hz, 2H); 2.27 (s, 3H); 1.95˜1.91 (m, 1H); 0.90 (d, J=6.61 Hz, 6H).

### EXAMPLE 64

### N-(1-Benzyl-2-oxo-2-{4-[5-(2-oxo-hexahydro-thieno[3,4-d]imidazol-6-yl)-pentanoyl]-piperazin-1-yl}-ethyl)-4-(5,6,7-trihydroxy-8-isobutyl-2-methyl-4-oxo-4H-chromen-3-yl)-benzamide

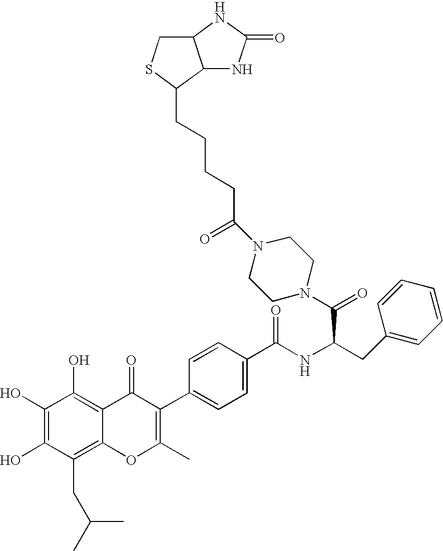

¹H NMR (DMSO-d₆, 300 MHz), δ 12.75 (br, 1H), 9.86 (br, 1H); 8.90 (m, 1H); 7.89 (d, J=7.75 Hz, 2H); 7.42 (d, J=7.74 Hz, 2H); 7.34˜7.22 (m, 5H); 6.44 (br, 2H); 5.12 (br, 1H); 4.37˜2.60 (m, 16H); 2.56 (d, J=6.93 Hz, 2H); 2.60-2.30 (m, 2H); 2.27 (s, 3H); 1.93 (m, 1H); 1.60˜1.20 (m, 6H); 0.90 (d, J=6.00 Hz, 6H).

### EXAMPLE 65

### 3-Phenyl-2-[4-(5,6,7-trihydroxy-8-isobutyl-2-methyl-4-oxo-4H-chromen-3-yl)-benzoylamino]-propionic acid

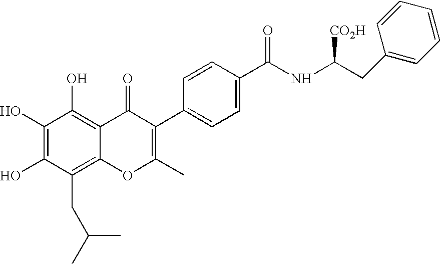

¹H NMR (DMSO-d₆, 300 MHz), δ 12.75 (s, 1H); 9.88 (s, 1H); 9.13 (s, 1H); 8.78 (m, 1H); 7.85˜7.15 (m, 9H); 4.64 (m, 1H); 3.41˜3.09 (m, 2H); 2.59 (d, J=7.00 Hz, 2H); 2.27 (s, 3H); 1.93 (m, 1H); 0.90 (d, J=6.40 Hz, 6H).

### EXAMPLE 66

### 5,6,7-Trihydroxy-8-isobutyl-2-methyl-3-[4-(4-phenyl-piperazine-1-carbonyl)-phenyl]-chromen-4-one

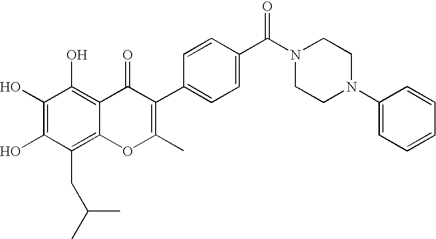

¹H NMR (DMSO-d₆, 300 MHz), δ 12.74 (br, 1H); 9.86 (br, 1H); 7.54˜6.95 (m, 9H); 4.07˜3.34 (m, 8H); 2.60 (d, J=6.90 Hz, 2H); 2.31 (s, 3H); 1.98˜1.90 (m, 1H); 0.90 (d, J=6.60 Hz, 6H); ¹³C NMR (DMSO-d₆, 75 MHz), δ 181.18; 169.57; 164.74; 152.62; 148.55; 145.76; 134.47; 131.48; 129.97; 129.00; 127.55; 119.82; 118.02; 106.34; 103.65; 31.89; 31.65; 28.71; 25.46; 23.14; 22.75; 20.02; 14.66.

### EXAMPLE 67

### 3-[4-(4-Benzyl-piperidine-1-carbonyl)-phenyl]-5,6,7-trihydroxy-8-isobutyl-2-methyl-chromen-4-one

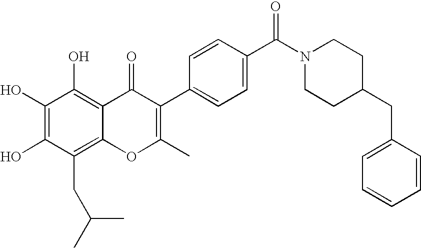

¹H NMR (DMSO-d₆, 300 MHz), δ 12.75 (s, 1H); 9.86 (br, 1H); 9.13 (br, 1H); 7.43˜7.13 (m, 9H); 4.46˜2.90 (m, 4H); 2.61˜2.51 (m, 4H); 2.31 (s, 3H); 1.98˜1.81 (m, 2H); 1.80˜1.15 (m, 4H); 0.90 (d, J=6.64 Hz, 6H); ¹³C NMR (DMSO-d₆, 75 MHz), δ 181.34; 169.56; 164.90; 152.80; 148.74; 145.89; 140.89; 136.47; 134.20; 131.57; 129.89; 129.05; 127.28; 126.71; 120.04; 106.49; 103.81; 42.94; 39.78; 38.38; 31.82; 28.88; 23.31; 22.93; 20.18; 14.84.

### EXAMPLE 68

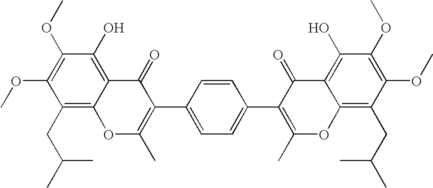

¹H NMR (CDCl₃, 300 MHz), δ 12.83 (b, 2H), 7.34 (s, 4H), 4.04 (s, 6H), 3.93 (s, 6H), 2.65 (d, J=7.2 Hz, 4H), 2.39 (s, 6H), 1.94 (m, 2H), 0.96 (d, J=6.6 Hz, 12H); ¹³C NMR (CDCl₃, 75 MHz), δ 181.88, 164.59, 157.54, 152.24, 150.34, 136.32, 131.69, 130.62, 120.79, 112.59, 107.04, 61.30, 60.66, 32.0, 29.09, 22.67, 19.74.

### EXAMPLE 69

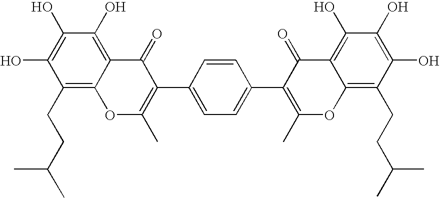

¹H NMR (CO(CD₃)₂, 300 MHz), δ 8.8 (b, 2H), 7.42 (s, 4H), 2.86 (m, 4H), 2.43 (s, 6H), 1.72 (m, 2H), 1.49 (m, 4H), 0.99 (d, J=7.0 Hz, 12H); ¹³C NMR (CO(CD₃)₂, 75 MHz), δ 176.31, 161.50, 155.71, 151.44, 151.09, 143.97, 132.50, 130.56, 123.31, 120.74, 114.91, 39.28, 28.36, 22.60, 21.61, 19.37.

### EXAMPLE 70

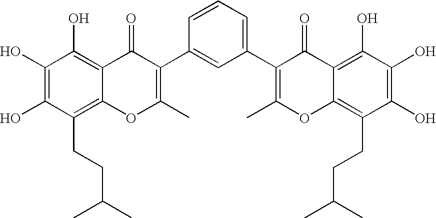

¹H NMR (CO(CD₃)₂, 300 MHz), δ 8.6 (b, 2H), 7.56 (t, J=7.0 Hz, 1H), 7.42 (t, J=1.5 Hz, 1H), 7.38 (m, 2H), 2.85 (t, J=7.7 Hz, 4H), 2.44 (s, 6H), 1.61 (m, 2H), 1.53 (m, 4H), 0.99 (d, J=6.4 Hz, 12H); ¹³C NMR (CO(CD₃)₂, 75 MHz), δ 181.09, 164.21, 150.43, 148.24, 144.64, 133.19, 132.49, 130.05, 127.74, 120.26, 106.94, 103.82, 38.36, 22.02, 20.19, 18.79.

### EXAMPLE 71

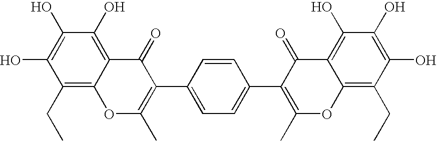

¹H NMR (CO(CD₃)₂, 300 MHz), δ 7.44 (s, 4H), 2.86 (q, J=7.4 Hz, 4H), 2.42 (s, 6H), 1.22 (t, J=7.4 Hz, 6H); ¹³C NMR (CO(CD₃)₂, 75 MHz), δ 181.13, 167.16, 151.23, 149.16, 145.21, 132.13, 131.24, 128.34, 121.04, 106.78, 104.23, 21.41, 18.42, 16.43.

### EXAMPLE 72

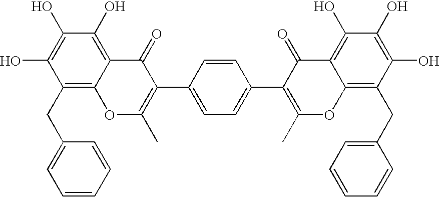

¹H NMR (CD₃OD, 300 MHz), δ 7.29-7.32 (m, 12H), 7.20 (m, 2H), 4.23 (s, 4H), 2.33 (s, 6H); ¹³C NMR (CD₃OD, 75 MHz), δ 175.95, 161.39, 155.66, 151.71, 151.28, 143.78, 140.24, 132.28, 130.33, 128.41, 125.96, 123.17, 118.43, 114.70, 31.23, 20.15.

### EXAMPLE 73

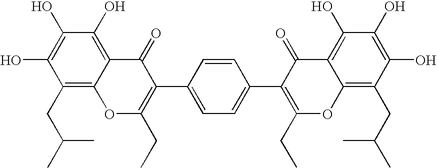

¹H NMR (CO(CD₃)₂, 300 MHz), δ 7.45 (s, 4H), 2.73-2.77 (m, 8H), 2.06 (m, 2H), 1.34 (t, J=7.5 Hz, 6H), 0.99 (d, J=6.7 Hz, 12H); ¹³C NMR (CO(CD₃)₂, 75 MHz), δ 176.43, 165.16, 155.96, 151.54, 151.11, 143.61, 132.28, 130.32, 122.48, 119.20, 114.56, 32.42, 28.92, 25.82, 22.62, 16.74.

### EXAMPLE 74

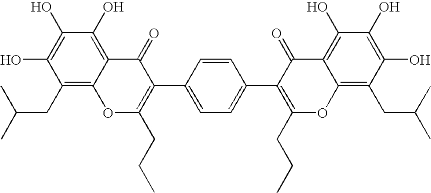

¹H NMR (CO(CD₃)₂, 300 MHz), δ 7.46 (s, 4H), 2.71-2.77 (m, 8H), 2.06 (m, 2H), 1.84 (hex, J=7.4 Hz, 4H), 0.99 (d, J=6.6 Hz, 12H), 0.96 (t, J=7.4 Hz, 6H); ¹³C NMR (CO(CD₃)₂, 75 MHz), δ 176.47, 164.17, 156.0, 151.57, 151.14, 143.65, 132.31, 130.45, 123.29, 119.23, 114.61, 34.03, 32.49, 28.99, 22.68, 20.47, 13.66.

### EXAMPLE 75

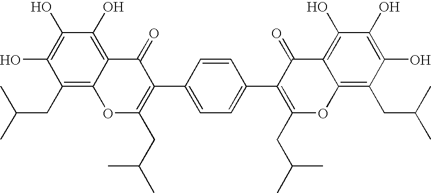

¹H NMR (CO(CD₃)₂, 300 MHz), δ 7.43 (s, 4H), 2.73 (t, J=7.2 Hz, 4H), 2.61 (d, J=7.3 Hz, 4H), 2.23 (m, J=6.8 Hz, 2H), 2.04 (m, 2H), 0.90-0.99 (m, 4H); ¹³C NMR (CO(CD₃)₂, 75 MHz), δ 176.38, 163.59, 155.89, 151.49, 151.04, 143.54, 132.21, 130.48, 123.87, 119.23, 114.48, 40.78, 32.40, 28.88, 26.88, 22.56, 2.25.

### EXAMPLE 76

### Modeling of the Binding of Gossypol and Analogs to Bcl-xL

The binding of gossypol to Bcl-xL was determined using ¹⁵N Heteronuclear Single Quantum Coherence Spectroscopy (HSQC) NMR methods. The protein samples for NMR studies were uniformly labeled with ¹⁵N for screening and uniformly double labeled with ¹⁵N and ¹³C for structure characterization according to the methods described in Jansson et al., J. Biomol. NMR, 7:131 (1996), and Cai et al., J. Biomol. NMR, 11:97 (1998). Since the NMR experiments were performed at pH 7.2 in a pulse field gradient (PFG), HSQC with water flip back was used to maximize signal intensity (Grzesiek et al., J. Am. Chem. Soc., 115:12593 (1993); and Sheppard et al., Abstracts of Papers of the Amer. Chem. Soc., 213:81 (1997)) and to minimize destruction from the water signal. HSQC spectra of Bcl-xL were recorded prior to (free Bcl-xL) and after the addition of the concentrated inhibitor solution. The two spectra were compared to identify the chemical shifts induced by the additions of the inhibitor. Data processing was conducted using nmrPipe, pipp and nmrDraw software (See, Garrett et al., J. Magn. Reson. Ser. B, 95:214 (1991); and Delaglio et al., J. Biomol. NMR, 6:277 (1995)). Shifted peaks were cross-referenced to the assignment table to reveal the residues affected by the presence of gossypol compounds.

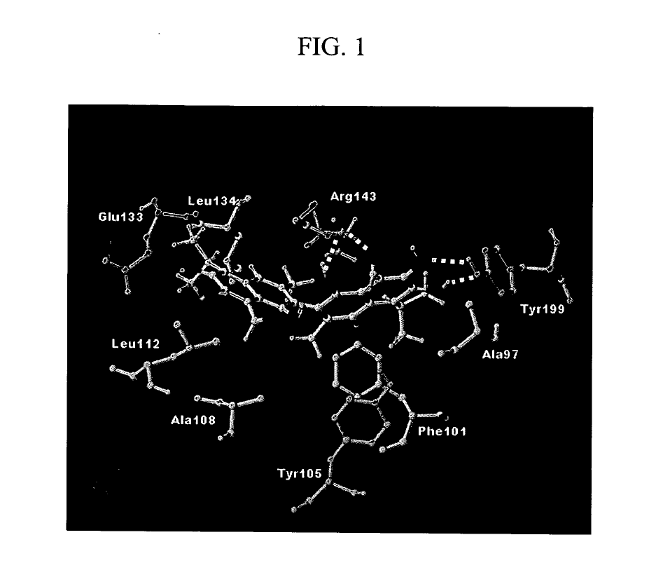

The 3-D NMR spectrum of the gossypol/Bcl-xL complex revealed that gossypol binds to the surface pocket on the Bcl-xL protein where BH3 domains of pro-apoptotic proteins bind (FIG. 1). A close inspection of the gossypol/Bcl-xL complex structure revealed several crucial interactions between these two molecules. One half of the gossypol molecule (the one on the right in FIG. 1) occupies the cavity primarily defined by Phe 101, Leu 103, Tyr 105, Gly 142, Arg 143, Ile 145, and Tyr 199. The multiple hydroxyl groups and the aldehyde group form a hydrogen bonding network with Arg 143 and Tyr 199; while the naphthyl ring together with the hydrophobic substituent groups (an isopropyl and a methyl group) on it fits into the hydrophobic bottom of this cavity. For the other half of gossypol (the one on the left in FIG. 1), the naphthyl ring acts as a huge hydrophobic group and fits into the cavity formed by Ala 108, Leu 112, Leu 134, and Ala 146.

Based on these observations, 5,6,7-trihydroxy-3-(6-hydroxy-5-isopropyl-naphthalen-2-yl)-8-isobutyl-2-methyl-chromen-4-one (compound 2) was designed to mimic the interaction between gossypol and Bcl-2 and Bcl-xL. It is clear that compound 2 shares a similar structural geometry with gossypol. The ketone group at the 4-position in compound 2 plays the same role in interacting with Bcl-xL as the 1-hydroxyl group in gossypol. Compared with the structure of gossypol, several other modifications have also been made. The aldehyde group on gossypol (which may be responsible for in vivo toxicity) was replaced by a hydroxyl group. The molecular modeling showed that this hydroxyl group may also be able to form the crucial hydrogen bond with Arg 143 on Bcl-xL. Analysis of the gossypol/Bcl-xL complex structure revealed that a moderately larger hydrophobic group could be accommodated in the cavity where the isopropyl group on gossypol binds (the one on the right in FIG. 1), so it was replaced by a relatively larger isobutyl group. Because the left half of gossypol acts as a large hydrophobic group fitting into the left hydrophobic pocket (FIG. 1), all of the undesired substituted groups were removed. These modifications also make it is more feasible to synthesize the designed compounds. In order to investigate the crucial factors for binding affinity, some other modifications on the left naphthyl ring and the core structure were also made.

The structure-based modeling revealed that compound 2 closely mimics gossypol to achieve the interaction with Bcl-xL. The carbonyl group together with the other three hydroxyl groups on the isoflavone moiety form the hydrogen bonding network with residue Arg139 and Tyr195 while the hydrophobic side of this moiety touches the hydrophobic bottom of the binding cavity. Overall, this half of compound 2 resembles closely the binding mode of gossypol. As for the naphthyl moiety, it fits into the hydrophobic cavity formed by Ala 104, Leu 108, Leu 112, Leu 130, and Ala 142. Compared to gossypol, compound 2 lacks one methyl group on the naphthyl moiety, which makes the relative rotation of its two major parts possible. As a result, compound 2 is more flexible than gossypol and its naphthyl moiety may fit better into the hydrophobic cavity formed by Ala 104, Leu 108, Leu 112, Leu 130, and Ala 142 and yet stays in an energetically favorable conformation.

### EXAMPLE 77

### Binding and Cellular Activity

Based on the NMR study of binding between gossypol and Bcl-xL, followed by computational structure-based modeling, isoflavone analogues (compounds 15a-15e and 15j) were designed and synthesized as novel Bcl2/Bcl-xL inhibitors.

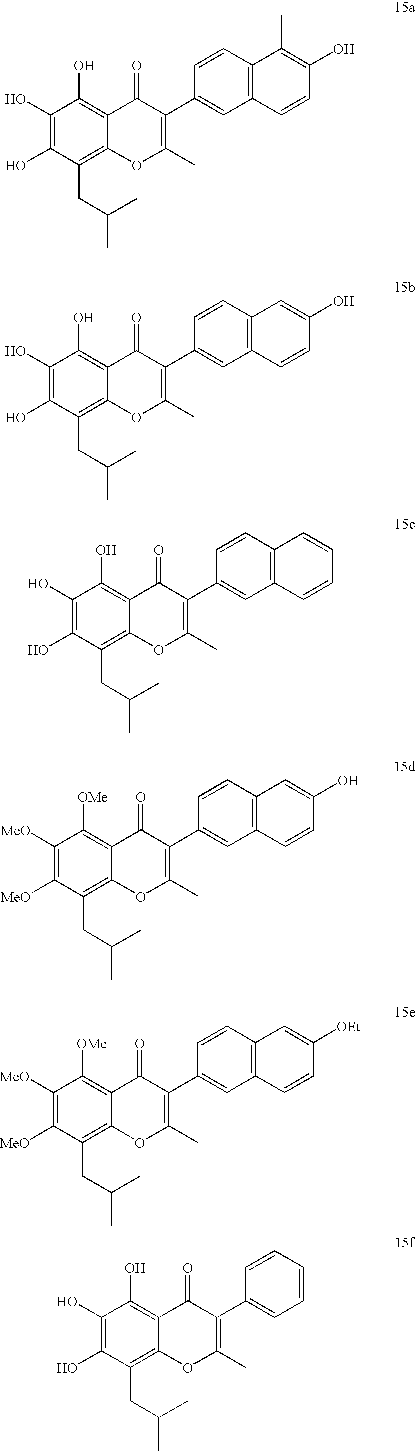

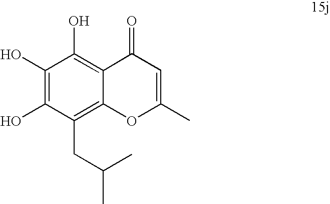

The binding affinities of these compounds and compound 2 with Bcl-2 and Bcl-xL were determined by a fluorescence polarization-based binding assay.

Bcl-2 Binding Assay

A 21-residue Bid BH3 peptide (QEDIIRNIARHLAQVGDSMDR) (SEQ ID NO:1) labeled at the N-terminus with 6-carboxyfluorescein succinimidyl ester (FAM) was used as the fluorescent tag (Flu-Bid-21). It was shown that this fluorescent peptide has high binding affinity with a K_{d} of 15.74 nM. Bcl-2 used in this assay is a recombinant His-fused soluble protein.

A 5 μl sample of the test compound dissolved in DMSO and preincubated Bcl-2 protein (0.120 μM) with Flu-Bid-21 peptide (0.010 μM) in assay buffer (100 mM potassium phosphate, pH 7.5; 100 μg/ml bovine gamma globulin; 0.02% sodium azide, purchased from Invitrogen Corporation, Life Technologies), are added in Dynex 96-well, black, round-bottom plates (Fisher Scientific) to produce a final volume of 125 μl. For each assay the bound peptide control containing Bcl-2 and Flu-Bid-21 peptide (equivalent to 0% inhibition), and free peptide control containing only free Flu-Bid-21 (equivalent to 100% inhibition), are included on each assay plate. The polarization values in millipolarization units (mP) are measured at excitation wavelength at 485 nm and an emission wavelength at 530 nm, after 4 hours incubation when the binding reached equilibrium, using the Ultra plate reader (Tecan U.S. Inc., Research Triangle Park, NC). IC₅₀, the inhibitor concentration at which 50% of bound peptide is displaced, is determined from the plot using nonlinear least-squares analysis and curve fitting using GraphPad Prism® software. The unlabeled Bid peptide is used as the positive control. The Kᵢ values were calculated using our developed equation for FP assay (Nikolovska-Coleska et al., Anal. Biochem. 332:261 (2004)). The program for calculating a Kᵢ value is available free of charge via the Internet at http://sw16.im.med.umich.edu/software/calc_ki/.

Bcl-xL Binding Assay

For determination of the binding affinity to Bcl-xL protein a human Bcl-xL recombinant His-tagged protein without the C-terminus hydrophobic tail and the Bak-16mer BH3 peptide labeled with 6-carboxyfluorescein succinimidyl ester (FAM) were used. This peptide has shown binding affinity of K_{d}=9.79 nM. The competitive binding assay was performed in the same way as for Bcl-2 protein using a preincubated complex with 60 nM Bcl-xL and 5 nM Flu-Bak peptide in assay buffer containing 50 mM Tris-Bis, pH 7.4; 0.01% bovine gamma globulin.

The binding affinity of the isoflavone analogs is shown in Table 2.

TABLE 2

| | Binding Affinity | | Cellular Activity | |
|---|---|---|---|---|
| | Kᵢ (μM) | | IC₅₀ (μM) | |
| Compound | Bc1-xL | Bc1-2 | PC3 | 2LMP |
| 2 | 1.49 ± 0.18 | 0.088 ± 0.003 | 1.82 | 1.54 |
| 15a | 1.78 ± 0.13 | 0.13 ± 0.01 | 3.09 | 1.44 |
| 15b | 1.71 ± 0.22 | 0.17 ± 0.02 | 5.07 | 2.55 |
| 15c | 2.32 ± 0.26 | 0.39 ± 0.17 | 3.62 | 2.08 |
| 15d | >14 | 1.24 ± 0.13 | 20.61 | >30 |
| 15e | >14 | >12 | >40 | >30 |
| 15f | 4.27 ± 0.84 | 0.79 ± 0.03 | 23.7 | 9.3 |
| 15j | >14 | 4.63 ± 0.16 | 20.1 | >30 |
| gossypol | 1.97 ± 0.39 | 0.23 ± 0.05 | 9.7 | 6.00 |

To test the effect of compounds of the present invention on inhibition of cell growth in human cancer cells, the compounds were administered to two different cancer cell lines. PC-3 prostate cancer cells and 2LMP breast cancer cells were each seeded in 96-well plates with increasing concentrations of inhibitor compounds. The cells were then incubated at 37° C. with 5% CO₂ for 5 days, followed by detection of cell viability with MTT. Untreated cells were used as 100% growth. The cell growth inhibition results are shown in Table 1.

Because of the highly structural similarity with gossypol, compound 2 showed extremely similar binding affinity to Bcl-xL (Kᵢ value 1.49 μM) and Bcl-2 (Kᵢ value 0.088 μM) with gossypol. In terms of cell growth inhibitory activity, compound 2 was 3 to 4-fold more potent than gossypol in both the 2LMP and PC3 cell lines.

When the isopropyl group of compound 2 was replaced by a smaller group such as methyl group or hydrogen (15a and 15b), there was little change in the binding affinity to Bcl-2 and Bcl-xL. These results showed that the isopropyl group in compound 2 made no contribution to the binding affinity to Bcl-2/Bcl-xL. These results confirmed previous NMR studies on gossypol that demonstrated that the isopropyl group on the left side (FIG. 1) was out of the binding pocket. The removal of the hydroxyl group in the 6′ position (15c) decreased the binding affinity of the compound to Bcl-2 about 2 fold, which indicated that this hydroxyl group might form hydrogen bonding interactions with a particular amino acid residue, although there was almost no change in binding affinity to Bcl-xL. The blockage of the hydroxyl groups in the core structure (15d) made the binding affinity to both proteins decrease dramatically (more than 10 fold, compare compound 15b with 15d). This demonstrated that the hydrogen bonding interaction between these hydroxyl groups and the protein (Arg 143 and Tyr 199 in Bcl-xL) is essential for the binding interaction. At the same time, the cellular growth inhibition activity on both the PC3 and 2LMP cell lines was decreased significantly which indicated that the cellular activities of the compounds correlated with their binding affinities to Bcl-2/Bcl-xL proteins. The blockage of the 6′ hydroxyl group in compound 15d with an ethyl group (15e) made the compound almost totally inactive (Kᵢ value or IC₅₀>10 μM) in both the binding and cellular assay.

Replacement of the substituted naphthyl ring with a smaller aromatic ring (phenyl group; 15f), or a hydrogen atom (15j) made the binding affinity decrease significantly. Especially in the monomer case (15j), the removal of the big hydrophobic group decreased the binding affinity more than 10-fold and 50-fold on Bcl-xL and Bcl-2 respectively, and at the same time the cellular activity was decreased. This suggested that a big hydrophobic group occupying the hydrophobic cavity on the surface of the proteins is also essential for the binding affinity.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

What is claimed is:

1. A compound having formula I: 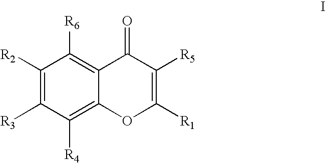
or a pharmaceutically acceptable salt or prodrug thereof, wherein:
R₁ is H, OH, F, Cl, Br, I, or optionally substituted alkyl, cycloalkyl, alkenyl, cycloalkenyl, alkynyl, aryl, heteroaryl, or heterocyclic;
R₂, R₃, R₄, R₅, and R₆ are independently H, F, Cl, Br, I, OH, or optionally substituted alkyl, cycloalkyl, alkenyl, cycloalkenyl, alkynyl, aryl, heteroaryl, heterocyclic, CO₂R′, C(O)NR′R″, SO₂NR′R″, SR′, OR′, NR″C(O)R′, NR′SO₂R″, or NR′R″;
R′ and R″ are independently H or optionally substituted alkyl, cycloalkyl, alkenyl, cycloalkenyl, alkynyl, aryl, heteroaryl, or heterocyclic, or R′ and R″ together with the N to which they are attached form a heterocyclic or heteroaryl ring.

2. The compound of claim 1, having Formula II: 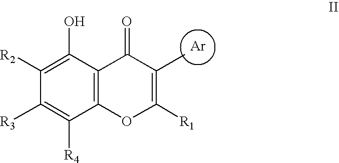
or a pharmaceutically acceptable salt or prodrug thereof, wherein:
Ar is optionally substituted aryl or heteroaryl.

3. The compound of claim 1, having Formula III: 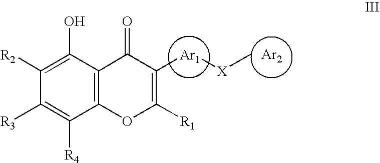
or a pharmaceutically acceptable salt or prodrug thereof, wherein:
Ar₁ and Ar₂ are independently optionally substituted aryl or heteroaryl;
X is O, N′, SO₂, S, C(O)N(R′), SO₂NR′, R′NCO, R′NSO₂, N(R′)R″, N(R′)—R″—N(R′″), R′, OR′, OR′O, or C(O)N(R′)R″; and
R′, R″, and R′″ are independently H or optionally substituted alkyl, cycloalkyl, alkenyl, cycloalkenyl, alkynyl, aryl, heteroaryl, or heterocyclic, or two of R′, R″, and R′″ form a heterocyclic or heteroaryl ring.

4. The compound of claim 2, having Formula IV: 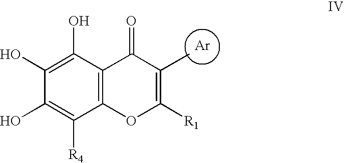
or a pharmaceutically acceptable salt or prodrug thereof.

5. The compound of claim 3, having Formula V: 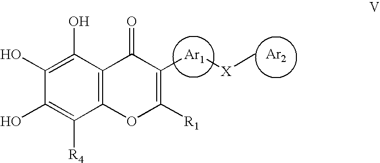
or a pharmaceutically acceptable salt or prodrug thereof.

6. The compound of claim 1, having Formula VI: 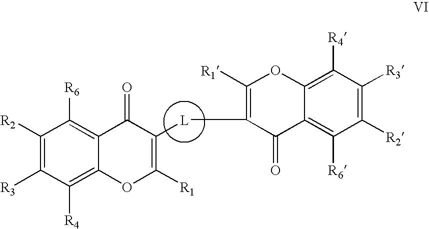
or a pharmaceutically acceptable salt or prodrug thereof; wherein
L is optionally substituted aryl, bi-aryl, heteroaryl, heterocyclic, alkyl, cycloalkyl, alkenyl, cycloalkenyl, alkynyl, ether, ester, amine, amide, sulfonyl, sulfonamide, or thioether;
R₁ and R₁′ are independently H, OH, F, Cl, Br, I, or optionally substituted alkyl, cycloalkyl, alkenyl, cycloalkenyl, alkynyl, aryl, heteroaryl, or heterocyclic; and
R₂, R₂′, R₃, R₃′, R₄, R₄′, R₆ and R₆′ are independently H, F, Cl, Br, I, OH, or optionally substituted alkyl, cycloalkyl, alkenyl, cycloalkenyl, alkynyl, aryl, heteroaryl, heterocyclic, CO₂R′, C(O)NR′R″, SO₂NR′R″, SR′, OR′, NR″C(O)R′, NR′SO₂R″, or NR′R″.

7. A pharmaceutical composition comprising a compound of claim 1 and a pharmaceutically acceptable carrier.

8. A method of inhibiting anti-apoptotic Bcl-2 family members in a cell comprising contacting the cell with a compound of claim 1.

9. A method of inducing apoptosis in a cell comprising contacting the cell with a compound of claim 1.

10. A method of rendering a cell sensitive to an inducer of apoptosis comprising contacting the cell with a compound of claim 1.

11. The method of claim 10, further comprising contacting the cell with an inducer of apoptosis.

12. The method of claim 11, wherein said inducer of apoptosis is a chemotherapeutic agent.

13. The method of claim 11, wherein said inducer of apoptosis is radiation.

14. A method of treating, ameliorating, or preventing a disorder responsive to the induction of apoptosis in an animal, comprising administering to said animal a therapeutically effective amount of a compound of claim 1.

15. The method of claim 14, further comprising administering an inducer of apoptosis.

16. The method of claim 15, wherein said inducer of apoptosis is a chemotherapeutic agent.

17. The method of claim 15, wherein said inducer of apoptosis is radiation.

18. The method of claim 14, wherein said disorder responsive to the induction of apoptosis is a hyperproliferative disease.

19. The method of claim 18, wherein said hyperproliferative disease is cancer.

20. The method of claim 15, wherein said compound of claim 1 is administered prior to said inducer of apoptosis.

21. The method of claim 15, wherein said compound of claim 1 is administered after said inducer of apoptosis.

22. The method of claim 15, wherein said compound of claim 1 is administered concurrently with said inducer of apoptosis.

23. A kit comprising a compound of claim 1.

24. The kit of claim 23, further comprising instructions for administering said compound to an animal.

25. The kit of claim 23, further comprising an inducer of apoptosis.

26. The kit of claim 25, wherein said inducer of apoptosis is a chemotherapeutic agent.

27. The kit of claim 24, wherein said instructions are for administering said compound to an animal having a hyperproliferative disease.

28. The kit of claim 27, wherein said hyperproliferative disease is cancer.
